SEQUENCE LISTING (26 sequences)

SEQ ID NO: 1 - DNA, 1073 bp, Artificial Sequence
  Oct4
atggccggccacctggccagcgatttcgccttcagccctccacctggcggaggcggagatggacctggcggccctgaacctggctgggtggaccctcggacctggctgagctttcagggccctccaggcggacctggaattggccctggcgtgggccctggatctgaagtgtggggcatccctccctgccccccaccctacgagttttgcggcggcatggcctactgtggccctcaggtcggagtgggactggtgcctcagggcggcctggaaacctctcagcctgagggcgaagccggcgtcggcgtggagagcaactctgacggagccagccctgagccttgtaccgtgacccctggcgccgtgaagctggaaaaagagaagctggaacagaaccccgaggaaagccaggacatcaaggccctgcagaaagaactggaacagttcgccaagctgctgaagcagaagcggatcacactgggatacacccaggccgatgtgggcctgaccctgggcgtgctgttcggcaaggtgttcagccagaccaccatctgcagattcgaagccctgcagctgagcttcaagaacatgtgcaagctgcggcccctgctgcagaaatgggtggaggaagccgacaacaacgagaacctgcaggaaatctgcaaggccgagacactggtgcaggcccggaagcggaagcggaccagcatcgagaacagagtgcggggcaacctggaaaacctgttcctgcagtgccccaagcccaccctgcagcagatcagccacattgctcagcagctcggcctggaaaaggacgtcgtgagagtgtggttctgcaaccggcggcagaagggcaagcggagcagcagcgactacgcccagagagaggacttcgaggccgctggcagcccttttagcggcggacccgtgtcctttcctctggcccctggccctcactttggcacccctggctacggcagcccacacttcaccgccctgtacagcagcgtgccctttccagagggcgaggccttcccccctgtgtccgtgaccaccctgggcagccccatgca

SEQ ID NO: 2 - DNA, 943 bp, Artificial Sequence
  Sox-2
gcatgtacaacatgatggaaaccgagctgaagcctcccggccctcagcagacaagtggaggcggcggaggcaattctacagccgccgctgccggcggaaaccagaagaacagccccgacagagtgaagcggcccatgaacgccttcatggtctggtccagaggacagaggcggaagatggcccaggaaaaccccaagatgcacaacagcgagatcagcaagagactgggcgccgagtggaagctgctgagcgagacagagaagcggcccttcatcgacgaggccaagcggctgagagccctgcacatgaaggaacaccccgactacaagtaccggcccagaagaaagaccaagaccctgatgaagaaggacaagtacaccctgccaggcggactgctggccccaggcggcaattctatggccagcggagtgggagtgggagctggactgggagccggcgtgaaccagcggatggacagctacgcccacatgaacggctggtccaacggcagctacagcatgatgcaggaccagctgggctaccctcagcaccctggcctgaatgcccatggcgccgctcagatgcagcccatgcaccgctacgatgtgtccgccctgcagtacaacagcatgaccagcagccagacctacatgaatggcagccccacctacagcatgtcctacagccagcagggcacaccaggcatggccctgggctctatgggcagcgtggtcaagagcgaggccagcagcagccctcctgtggtcaccagcagctcccacagcagagccccttgtcaggccggcgacctgcgggacatgatcagcatgtacctgcctggcgccgaagtgcctgaacctgccgcccctagcagactgcacatgagccagcactaccagagcggccctgtgcctggcaccgccatcaatggcaccctgcccct

SEQ ID NO: 3 - DNA, 1417 bp, Artificial Sequence
  Flf4
tctggcatggccgtgagcgacgccctgctgcccagcttcagcacctttgccagcggacctgcaggcagagagaaaaccctgcggcaggctggcgcccctaacaaccggtggcgggaggaactgtcccacatgaagagactgccccccgtgctgcccggcagaccttatgatctggccgctgccaccgtggccaccgatctggaatctggcggagccggcgctgcctgtggcggaagcaacctggcccccctgcccagacgggagacagaagagttcaacgacctgctggacctggacttcatcctgagcaacagcctgacccaccctcctgagtctgtggccgccaccgtgtctagcagcgccagcgccagcagctctagctctcctagctcttctggccctgccagcgcccccagcacctgtagcttcacctaccccatcagagccggcaatgatcctggcgtggcaccaggcggaacaggcggaggactcctgtacggcagagagtctgccccaccccccaccgcccccttcaacctggccgacatcaacgacgtgtcccccagcggaggctttgtggccgagctgctgaggcctgagctggaccccgtgtacatccccccacagcagcctcagcctccaggcggcggactgatgggcaagttcgtgctgaaagccagcctgagcgcccctggctctgagtatggctcccccagcgtgatcagcgtgtccaagggcagccctgatggctctcaccctgtggtggtggccccttacaatggcggccctcccagaacctgccccaagatcaagcaggaagccgtcagcagctgtacacacctgggcgctggccctccactgagcaacggacacaggcctgccgcccacgactttccactgggcagacagctccctagcagaaccacccccaccctggggctggaagaggtgctgagcagcagagactgccaccctgccctgcctctgccacctggctttcatcctcacccaggccccaactaccccagcttcctgcccgaccagatgcagcctcaggtgccccccctgcactaccaggaactgatgcccccaggcagctgcatgcccgaggaacccaagcccaagcggggcagaagaagctggccccggaagagaaccgccacccacacctgtgattacgccggctgcggcaagacctacaccaagagcagccacctgaaggcccacctgagaacccacaccggcgagaagccctaccactgcgactgggacggctgcggctggaagttcgccagaagcgacgagctgacccggcactacagaaagcacaccggccaccggcccttccagtgccagaagtgcgaccgggccttcagcagatccgaccacctggctctgcatatgaagcggcactttt

SEQ ID NO: 4 - DNA, 69 bp, Artificial Sequence
  miRNA302A
ccaccacttaaacgtggatgtacttgctttgaaactaaagaagtaagtgcttccatgttttggtgatgg

SEQ ID NO: 5 - DNA, 68 bp, Artificial Sequence
  miRNA302D
cctctactttaacatggaggcacttgctgtgacatgacaaaaataagtgcttccatgtttgagtgtgg

SEQ ID NO: 6 - DNA, 68 bp, Artificial Sequence
  miRNA367
ccattactgttgctaatatgcaactctgttgaatataaattggaattgcactttagcaatggtgatgg

SEQ ID NO: 7 - DNA, 4374 bp, Artificial Sequence
  BDD-FVIII
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcaccgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 8 - DNA, 2538 bp, Artificial Sequence
  VEC promoter
  misc_feature (580)..(580): n is a, c, g, or t
ctagtagcagaaacaaggtcctctggaagagcaactgatgctcttaggtactgaagcatcatcctgccccagagaccactcgcatatgaagcacacatattcagtctgccttacttgtgttaatgattgccagtgtccctctgacctcctagccctgaaaaggtgtggcctgaaggtcatttcagagacggggagagctgctcagagaagccaatcggcgagtctaggacacacagacaggatctagtcccagagttcgctagcctaggtgagcgtcccctggccccttataccacttccttctccagcttgcatctaattcgctctggcagaccatcgtgtttcctgtcttcctggcagcctccagcacgctcagtgctactccctcgcatgcgccctcctcccagtaccttctctgactccagtgggcttggagtgcgaggaggaagggtgaggaaggggtgaaatcaggtattggatccacagggggtctgaagagcactagcctggccttttgggactgaacttctgctatgaagacctccactgccatccctggagtccggggcacatccaaggnttgctgtccatcgtttaactgtttacagatgacaacaatgactcgtgttcggggcagaaatatcaccagggctagagtacaaaaggagtttgcattgatggccggacaggcctgtccctggaccagcctgcgacgctgagtatgagacccagcggaagtgctaccctggcagacgtgtcactgagtacacagaccaccaaggcaggcagctctcggggaagctgtctatgctgggccagcccaccttgagggcagggaacagaacagattgtggcagagaggaaaatgtggagcttctgtttgttcacagacacacgcactcgcccacgcacgcacgcacgcacgcacgcacgcacgaatgcacgcacgcagtagttgaatgctatggattccgctcagagctgagaacagccccagcgacagttccctggcctctctccttactctgatgtcctcatctgtcttcacatggtctcaggacgctaatactccatcctaatgtacactcctttccctgggcctccgttccagttcagttctcagaggacctggagggagtgattggctacaccaactttgctttcgttcaccaagcccatgtctctacttgggtgtctaatgggcatctccaacattacctaccccaaacagaaaaccctttcttccccccaaccacaccccaccctacccccacagtattttctccatgcccggaaagatctgctctcttatggtccctctttgcctcactgaaaagcaggacaagttggggaacttcccaaacttttatgcatgaagaaacccaggcaatttgccaaaaggtacactctgggggtctgtcatttactctgagccagaaccctgaaatttttactaacccatcacataatgaatgaagagaatctttttctttttttttttttttctttttttttggtttttcgagacagggtttctctgtatagccctggctatcctggaacacactctgtagaccaggctggcctcgaactcagaaatccacctgcctctgcctcccgagtgctgggattaaaggcgtgcgccaccacgcctggctgaatgaagagaatcttgacctcatctccccagcctcttggtcctgagggaccctggtctacctactgctttgctgtcttcttagctcttcttacttttttgctgactcagacctatggctatctccattatacagatgaggagactgaggcatggatccctggttggtccatggtcacgtgaagcccatcacccagtatttgtaaagtgagatgggccaggctggtaccttggaactgaaactcacactgccctacctggaagaatctgacaggcaaaatctgctgctgaaagtgattgtctgtcacgtttctcagctgcccgactctgagaactccacagccccctttcgttccaccatactacagagtcgccacggaaagccggctctgtggagaagctgaggtagctgggtttctgtctgggttactctgtccagcgaggaaacaagtaccttagacccactaagcctctgctttctgaactgtaaagtgggggatatgacacctgcctcccagggatggctgaatgctctggcagaagcttagagcccccacagctacccctaggctcacagctcctccgatgagacctagaattgaggtatgagttgaataccccaggcaggtccaaggcttccacgggcccaggctgaccaagctgaggccgcccaccgtagggcttgcctatctgcaggcagctcacaaaggaacaataacaggaaaccatcccgaggggaagtgggccagggcaagttggaaaacctgcctccctcccagcctgggtgtggctcccctctcccctcctgaggcaatcaactgtgctctccacaaagctcggccctggacagactcgactagaggatccaccggtcgccacca

SEQ ID NO: 9 - DNA, 1175 bp, Artificial Sequence
  LV.pF8.1
gagctcaccatggctacattctgatgtaaagagatatatcctatacctgggccaaatgtaaacagcctggaaaagtgttaggttaaaaacaaaacaaaataaataaatgaataaatgccaggtggttatgagtgctattgagaaaaatgaagccaagagggatatcagtgatgcaggtgggggtaaagagcttacaacataaatgtggtgttccatatttaaacctcattcaacagggaagattggagctgaaatgtgaaggagttgtgggagtggaactacgtggaaatctgggggaaaggtgttttgggtaaaagaaatagcaagtgttgaggtccaggggcatgagtgtgcttgatattttagggaagagtaaggagaccagtataaccagagtgagatgagactacagaggtcaggagaaagggcatgcagaccatgtgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 10 - DNA, 599 bp, Artificial Sequence
  LV.pF8.2
gtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 11 - DNA, 1698 bp, Artificial Sequence
  LV.pF8.3
ggggctcgctcgctcagtacctggaggcgagttcctgacgcgactgcgactcaatcctcgcctggtgaagaatattttacctatgactcactgaaaataaagacggctgagtgaccgtgtttgttcatgtaaacattgaacaaatatttatcggcttctgcgatgtgtcctactcttttagtggaggaagacacattttatttatgtatttaatttttcttttgaattttacatgcgagttatacttaataaaactcacttcaaaatataccttcaacagaaaatccagcaacagtttctattatgttagttaaaacagccagtcttttcctttacttttaaaaattattcataaatgtaattagtgaatgataataaacattgacatctgatccactgctttaggagtgacacaaatgaagttaactcaggctattttctttataatcattgtgctattgttttctttttcttttcaattatactgcttaatataggattttgtggcaccataggagttgaggagctcaccatggctacattctgatgtaaagagatatatcctatacctgggccaaatgtaaacagcctggaaaagtgttaggttaaaaacaaaacaaaataaataaatgaataaatgccaggtggttatgagtgctattgagaaaaatgaagccaagagggatatcagtgatgcaggtgggggtaaagagcttacaacataaatgtggtgttccatatttaaacctcattcaacagggaagattggagctgaaatgtgaaggagttgtgggagtggaactacgtggaaatctgggggaaaggtgttttgggtaaaagaaatagcaagtgttgaggtccaggggcatgagtgtgcttgatattttagggaagagtaaggagaccagtataaccagagtgagatgagactacagaggtcaggagaaagggcatgcagaccatgtgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 12 - DNA, 1122 bp, Artificial Sequence
  LV.pF8.4
ggggctcgctcgctcagtacctggaggcgagttcctgacgcgactgcgactcaatcctcgcctggtgaagaatattttacctatgactcactgaaaataaagacggctgagtgaccgtgtttgttcatgtaaacattgaacaaatatttatcggcttctgcgatgtgtcctactcttttagtggaggaagacacattttatttatgtatttaatttttcttttgaattttacatgcgagttatacttaataaaactcacttcaaaatataccttcaacagaaaatccagcaacagtttctattatgttagttaaaacagccagtcttttcctttacttttaaaaattattcataaatgtaattagtgaatgataataaacattgacatctgatccactgctttaggagtgacacaaatgaagttaactcaggctattttctttataatcattgtgctattgttttctttttcttttcaattatactgcttaatataggattttgtggcaccataggagttgaggtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 13 - DNA, 1979 bp, Artificial Sequence
  LV.pF8.5
tcgccaccacttggcttccggcacgtggggcagatgtttccattcccacggcggcagcggaagagggagggccgggcgcgccgcggctgcttgcagtctccgcaagcggctacatcacagagctcagcgtgcggtgtcacaggccccgcggtcccgcccaacagatgcaccgagatgcgcgtgcgcagaaagcgtcccgggggtgaggctccctccctcgctctccctctactcccgccccactctcccccactttcccccctccacccaccgcggccgtcggggctcgctcgctcagtacctggaggcgagttcctgacgcgactgcgactcaatcctcgcctggtgaagaatattttacctatgactcactgaaaataaagacggctgagtgaccgtgtttgttcatgtaaacattgaacaaatatttatcggcttctgcgatgtgtcctactcttttagtggaggaagacacattttatttatgtatttaatttttcttttgaattttacatgcgagttatacttaataaaactcacttcaaaatataccttcaacagaaaatccagcaacagtttctattatgttagttaaaacagccagtcttttcctttacttttaaaaattattcataaatgtaattagtgaatgataataaacattgacatctgatccactgctttaggagtgacacaaatgaagttaactcaggctattttctttataatcattgtgctattgttttctttttcttttcaattatactgcttaatataggattttgtggcaccataggagttgaggagctcaccatggctacattctgatgtaaagagatatatcctatacctgggccaaatgtaaacagcctggaaaagtgttaggttaaaaacaaaacaaaataaataaatgaataaatgccaggtggttatgagtgctattgagaaaaatgaagccaagagggatatcagtgatgcaggtgggggtaaagagcttacaacataaatgtggtgttccatatttaaacctcattcaacagggaagattggagctgaaatgtgaaggagttgtgggagtggaactacgtggaaatctgggggaaaggtgttttgggtaaaagaaatagcaagtgttgaggtccaggggcatgagtgtgcttgatattttagggaagagtaaggagaccagtataaccagagtgagatgagactacagaggtcaggagaaagggcatgcagaccatgtgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 14 - DNA, 1403 bp, Artificial Sequence
  LV.pF8.6
tcgccaccacttggcttccggcacgtggggcagatgtttccattcccacggcggcagcggaagagggagggccgggcgcgccgcggctgcttgcagtctccgcaagcggctacatcacagagctcagcgtgcggtgtcacaggccccgcggtcccgcccaacagatgcaccgagatgcgcgtgcgcagaaagcgtcccgggggtgaggctccctccctcgctctccctctactcccgccccactctcccccactttcccccctccacccaccgcggccgtcggggctcgctcgctcagtacctggaggcgagttcctgacgcgactgcgactcaatcctcgcctggtgaagaatattttacctatgactcactgaaaataaagacggctgagtgaccgtgtttgttcatgtaaacattgaacaaatatttatcggcttctgcgatgtgtcctactcttttagtggaggaagacacattttatttatgtatttaatttttcttttgaattttacatgcgagttatacttaataaaactcacttcaaaatataccttcaacagaaaatccagcaacagtttctattatgttagttaaaacagccagtcttttcctttacttttaaaaattattcataaatgtaattagtgaatgataataaacattgacatctgatccactgctttaggagtgacacaaatgaagttaactcaggctattttctttataatcattgtgctattgttttctttttcttttcaattatactgcttaatataggattttgtggcaccataggagttgaggtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 15 - DNA, 2350 bp, Artificial Sequence
  0 to 2350 5′ FVIII promoter sequence
cagcagttcccacaaacgttaccctcacaatgaatccagccatttttcaccctctccagtggtaccatcatagcccaagccgccaccatttctcacccccggttaacaggccaccctccttctacccttatcctgctagagtttgttttatctacagtgatcagaaagatcagcctaaaagataattctgatcaccaccctcctctactcacaacccggccgtgtctccccattgccctcagtgtagaagtcaatgtccctttgctgaaatgcaaccttagtgaaactttccatgactaacctcctttaaaattgcaacctggtccacccttactcccccttaccccacttctcttttttgcacagcacttattttaccttctaacatactgtataatgtactcatgtattgtaattattgcttatcatccctctttcagttgcttatatttttcatcaatgtgtacccagtgcctaggacaatatctgtctaggacaaatgggtagttatgtggctgtaggcaagccatttaacctctctgtacctcagttactttatctgtatccactttgcggtgttgtcatgaggattaaatcagatagcctatgtgtagcacctggcagtgaatttatcaccctgtactgtaactgtctacttttctgtctcctccattggactgtcattcccagggggttgggaactgggatttcttcatttctgaggcatagaagtatagcatagtggttaggagcatgacttctggagccagagtacatgggtttgaatgctaccactcacaagctgtgtggccatggagaagttgcctaacctctccgtgcttcagtttcatcacccataaaatgaaggtaagaatagtacctgtatttaaaagcacctagaacagttcctggcatatagtgtcagctgtcatctctgcatccttgtacctgtcagagaggagtgtttatcaaaggggcttcttgctgcctgtttccaaaccagtcgacaatataccaattgctccctaacacattcttgtttgtgcagaactgagctcaatgataacatttttatagcaaccctgatcaagtttcttctcataatctcttacactttgaggcccctgcaggggccctcactctccctaataaacattaacctgagtagggtgtttgagctcaccatggctacattctgatgtaaagagatatatcctatacctgggccaaatgtaaacagcctggaaaagtgttaggttaaaaacaaaacaaaataaataaatgaataaatgccaggtggttatgagtgctattgagaaaaatgaagccaagagggatatcagtgatgcaggtgggggtaaagagcttacaacataaatgtggtgttccatatttaaacctcattcaacagggaagattggagctgaaatgtgaaggagttgtgggagtggaactacgtggaaatctgggggaaaggtgttttgggtaaaagaaatagcaagtgttgaggtccaggggcatgagtgtgcttgatattttagggaagagtaaggagaccagtataaccagagtgagatgagactacagaggtcaggagaaagggcatgcagaccatgtgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 16 - DNA, 523 bp, Artificial Sequence
  Enhancer Short
ggggctcgctcgctcagtacctggaggcgagttcctgacgcgactgcgactcaatcctcgcctggtgaagaatattttacctatgactcactgaaaataaagacggctgagtgaccgtgtttgttcatgtaaacattgaacaaatatttatcggcttctgcgatgtgtcctactcttttagtggaggaagacacattttatttatgtatttaatttttcttttgaattttacatgcgagttatacttaataaaactcacttcaaaatataccttcaacagaaaatccagcaacagtttctattatgttagttaaaacagccagtcttttcctttacttttaaaaattattcataaatgtaattagtgaatgataataaacattgacatctgatccactgctttaggagtgacacaaatgaagttaactcaggctattttctttataatcattgtgctattgttttctttttcttttcaattatactgcttaatataggattttgtggcaccataggagttgag

SEQ ID NO: 17 - DNA, 804 bp, Artificial Sequence
  Enhancer Long
tcgccaccacttggcttccggcacgtggggcagatgtttccattcccacggcggcagcggaagagggagggccgggcgcgccgcggctgcttgcagtctccgcaagcggctacatcacagagctcagcgtgcggtgtcacaggccccgcggtcccgcccaacagatgcaccgagatgcgcgtgcgcagaaagcgtcccgggggtgaggctccctccctcgctctccctctactcccgccccactctcccccactttcccccctccacccaccgcggccgtcggggctcgctcgctcagtacctggaggcgagttcctgacgcgactgcgactcaatcctcgcctggtgaagaatattttacctatgactcactgaaaataaagacggctgagtgaccgtgtttgttcatgtaaacattgaacaaatatttatcggcttctgcgatgtgtcctactcttttagtggaggaagacacattttatttatgtatttaatttttcttttgaattttacatgcgagttatacttaataaaactcacttcaaaatataccttcaacagaaaatccagcaacagtttctattatgttagttaaaacagccagtcttttcctttacttttaaaaattattcataaatgtaattagtgaatgataataaacattgacatctgatccactgctttaggagtgacacaaatgaagttaactcaggctattttctttataatcattgtgctattgttttctttttcttttcaattatactgcttaatataggattttgtggcaccataggagttgag

SEQ ID NO: 18 - DNA, 4429 bp, Artificial Sequence
  Full 5′ FVIII promoter sequence
tcgccaccacttggcttccggcacgtggggcagatgtttccattcccacggcggcagcggaagagggagggccgggcgcgccgcggctgcttgcagtctccgcaagcggctacatcacagagctcagcgtgcggtgtcacaggccccgcggtcccgcccaacagatgcaccgagatgcgcgtgcgcagaaagcgtcccgggggtgaggctccctccctcgctctccctctactcccgccccactctcccccactttcccccctccacccaccgcggccgtcggggctcgctcgctcagtacctggaggcgagttcctgacgcgactgcgactcaatcctcgcctggtgaagaatattttacctatgactcactgaaaataaagacggctgagtgaccgtgtttgttcatgtaaacattgaacaaatatttatcggcttctgcgatgtgtcctactcttttagtggaggaagacacattttatttatgtatttaatttttcttttgaattttacatgcgagttatacttaataaaactcacttcaaaatataccttcaacagaaaatccagcaacagtttctattatgttagttaaaacagccagtcttttcctttacttttaaaaattattcataaatgtaattagtgaatgataataaacattgacatctgatccactgctttaggagtgacacaaatgaagttaactcaggctattttctttataatcattgtgctattgttttctttttcttttcaattatactgcttaatataggattttgtggcaccataggagttgagtaaaaataaaaggaataaaaatataccttatctggccgggcgcggtggctcacgcctgtaatttcagcagtttcggaggccgaggcgggcggatcacgcggtcaggagatcgaggccatcctggctaacatggtgaaaccccgtctctactaaaaatacaaaaaattagccgggcatggtggcggccgcctgtagtcccagctactcgggaggctgaggcaggagaatggcgtgaacccgggaggcggagcttgcagtgagccgagatcgcgacactgcactccagcctgggcgacagagtgagactgcgtctccaaaaaaaaaagaaaaaatacgttatctatgaagatttccaatttgatttctatttatcacaaatggccacagtactcctttgtactttaccacataccatattgtattcagtaattatttgtgaatatgtaattgataatattgtaggttttagagaatccttgaaaacatgaaaatttggtaatggggtctattttgattatttatttatttatttatttattttatttttgagacagagtctcgctcttgttgcccaggctggagtgcagtggcgcgatctcggctcactgcaagctccacctcccgggttcaagcgattctcctgcctcagcctcccaagtagctgggactacaggcacgtgccaccatgcccggctaattttttgtatttttagtagaggaggagtttcatcttgttagctaggatggtctagatctcctgacctcgtgatctgcccgcctcagcctcccaaagtgctgggattacaggtgtgagccaccgtgcccggccatattttgatttaaaatttagcaataatagataaaattttcaatcaactaagcccttgggccagggaatgctattccttaaaaagtgcttctatcaatatagcctctgactcattactttgttaatttttaaattgtatttcattcctgattaacattcccacccagattattaattatacaatctgttaactgtagaacctcaaacatgttggattgtactgtatttgtctggaagacacatttttaaaacattgtaatcgctataagagaagcactgggaaagaaaggagcttctatgcctgcagtgcctgaggagccctttaacagtgtgccccgcccctaagctactcatgcagtcatccccatcccagttagtcaactttattccaaaaaacttggtgttccaaatttttccttctcaaagcccacagatccaaaattcatcagcagttcccacaaacgttaccctcacaatgaatccagccatttttcaccctctccagtggtaccatcatagcccaagccgccaccatttctcacccccggttaacaggccaccctccttctacccttatcctgctagagtttgttttatctacagtgatcagaaagatcagcctaaaagataattctgatcaccaccctcctctactcacaacccggccgtgtctccccattgccctcagtgtagaagtcaatgtccctttgctgaaatgcaaccttagtgaaactttccatgactaacctcctttaaaattgcaacctggtccacccttactcccccttaccccacttctcttttttgcacagcacttattttaccttctaacatactgtataatgtactcatgtattgtaattattgcttatcatccctctttcagttgcttatatttttcatcaatgtgtacccagtgcctaggacaatatctgtctaggacaaatgggtagttatgtggctgtaggcaagccatttaacctctctgtacctcagttactttatctgtatccactttgcggtgttgtcatgaggattaaatcagatagcctatgtgtagcacctggcagtgaatttatcaccctgtactgtaactgtctacttttctgtctcctccattggactgtcattcccagggggttgggaactgggatttcttcatttctgaggcatagaagtatagcatagtggttaggagcatgacttctggagccagagtacatgggtttgaatgctaccactcacaagctgtgtggccatggagaagttgcctaacctctccgtgcttcagtttcatcacccataaaatgaaggtaagaatagtacctgtatttaaaagcacctagaacagttcctggcatatagtgtcagctgtcatctctgcatccttgtacctgtcagagaggagtgtttatcaaaggggcttcttgctgcctgtttccaaaccagtcgacaatataccaattgctccctaacacattcttgtttgtgcagaactgagctcaatgataacatttttatagcaaccctgatcaagtttcttctcataatctcttacactttgaggcccctgcaggggccctcactctccctaataaacattaacctgagtagggtgtttgagctcaccatggctacattctgatgtaaagagatatatcctatacctgggccaaatgtaaacagcctggaaaagtgttaggttaaaaacaaaacaaaataaataaatgaataaatgccaggtggttatgagtgctattgagaaaaatgaagccaagagggatatcagtgatgcaggtgggggtaaagagcttacaacataaatgtggtgttccatatttaaacctcattcaacagggaagattggagctgaaatgtgaaggagttgtgggagtggaactacgtggaaatctgggggaaaggtgttttgggtaaaagaaatagcaagtgttgaggtccaggggcatgagtgtgcttgatattttagggaagagtaaggagaccagtataaccagagtgagatgagactacagaggtcaggagaaagggcatgcagaccatgtgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 19 - DNA, 331 bp, Artificial Sequence
  miRNA let7b
ctgaggaccgccaggcaggggctggtgctgggcggggggcggcgggccctcccgcagtgcaaggccgggcctggcggggtgaggtagtaggttgtgtggtttcagggcagtgatgttgcccctcggaagataactatacaacctactgccttccctgaggagcccagtgacacgaccccatgggagggccgccccctacctcagtgacacgaccccacgggagggctgccccccacctcagtgacctgcagggggcctagccgaagctgggtgggcatctgggagctagattcaataaagctgttctgaccatgaacttggaactggcccc

SEQ ID NO: 20 - DNA, 717 bp, Artificial Sequence
  miRNA 126
gacggtacactctgtgtgcccaagggagggccccccagggtggcccccaacccgacaggtaaacagccctggctgtgcctggcctggggaggcgggcaggcagtggacattgccgtgtggctgttaggcatggtggggggcactggaatctgggcggaaggcggtggggactccctctccagggagggaggatggggagggaggataggtgggttcccgagaactgggggcaggttgcccggagcctcatatcagccaagaaggcagaagtgccccgtcccggggtcctgtctgcatccagcgcagcattctggaagacgccacgcctccgctggcgacgggacattattacttttggtacgcgctgtgacacttcaaactcgtaccgtgagtaataatgcgccgtccacggcaccgcatcgaaagcgccgctgagacctcagccttgacctccctcagcgtggccgggaccctgagcctctgcgcagagccacccgccccgacgtacttaggcggcatagccctgagacctctggccagcgccaggcaggcagcgggggcggcagaggcctgggcctgagtcttctggctctgcctctccctggggacaggagggagcctgggggtgtgggtggggagccggccggccgtgacccagcgcctggctctgcccgcaggagtggacagtgcaatgaaggaagaagtgcagaggctgca

SEQ ID NO: 21 - DNA, 20 bp, Artificial Sequence
  Wpre
tggattctgcgcgggacgtc

SEQ ID NO: 22 - DNA, 23 bp, Artificial Sequence
  dNEF
ggctaagatctacagctgccttg

SEQ ID NO: 23 - DNA, 21 bp, Artificial Sequence
  GAPDH
aacgtgtcagtggtggacctg

SEQ ID NO: 24 - DNA, 21 bp, Artificial Sequence
  GAPDH
agtgggtgtcgctgttgaagt

SEQ ID NO: 25 - DNA, 25 bp, Artificial Sequence
  Nanog promoter forward
tggttaggttggttttaaatttttg

SEQ ID NO: 26 - DNA, 25 bp, Artificial Sequence
  Nanog promoter reverse
acccacccttataaattctcaatta

(19) United States
(12) Patent Application Publication
(10) Pub. No.: US 2021/0340495 A1
(43) Pub. Date: Nov. 4, 2021

(54) METHOD FOR INDUCING AND DIFFERENTIATING PLURIPOTENT STEM CELLS AND USES THEREOF

(71) Applicant: Universita' del Piemonte Orientale, Vercelli (IT)

(72) Inventors: Antonia Follenzi, Novara (IT); Cristina Olgasi, Torino (IT); Maria Talmon, Pinasca-Dubbione (Torino) (IT)

(73) Assignee: Universita' del Piemonte Orientale, Vercelli (IT)

(21) Appl. No.: 16/322,640

(22) PCT Filed: Jul. 27, 2017
(86) PCT No.: PCT/IB2017/054574

§ 371 Date: Feb. 1, 2019

(30) Foreign Application Priority Data
Aug. 2, 2016 (IT) 102016000081180

(51) Int. Cl. C12N 5/071; A61K 35/44; C12N 15/86; C12N 5/074; A61K 35/545; A61K 45/06

(52) U.S. Cl. CPC C12N 5/069; C12N 2510/00; C12N 15/86; C12N 5/0696; A61K 35/545; A61K 45/06; C12N 2501/119; C12N 2501/165; C12N 2533/54; C12N 2501/155; C12N 2501/2303; C12N 2501/2307; C12N 2501/2306; C12N 2501/22; C12N 2501/145; C12N 2501/125; C12N 2501/26; C12N 2500/90; C12N 2740/15043; A61K 35/44

(57) ABSTRACT

The present invention refers to a method for inducing pluripotent stem cells starting from somatic cells isolated from healthy and/or diseased individuals. The diseased individual is preferably affected by a genetic disease such as type A hemophilia, and the somatic cells from the diseased individual are genetically corrected for the mutation causing the disease preferably after being reprogrammed by the method of the present invention. A further aspect of the present invention refers to a method for differentiating induced pluripotent stem cells or embryonic stem cell-like into endothelial cells. Moreover, the present invention refers to the use of these cells as a medicament for treating a disease, in particular, a genetic disease such as type A hemophilia.

The present invention refers to a method for inducing pluripotent stem cells starting from somatic cells isolated from healthy and/or diseased individuals, and to a method for differentiating induced pluripotent stem cells or embryonic stem cell-like into endothelial cells.

Moreover, the present invention refers to the use of these cells as a medicament for treating a disease, in particular, a genetic disease such as type A hemophilia.

### BACKGROUND

Induced pluripotent stem cells (iPSCs) are adult and/or somatic and/or differentiated cells that have been genetically reprogrammed to an embryonic stem cell-like state by being forced to express genes and factors important for maintaining the defining properties of embryonic stem cells (stemness genes). iPSCs self-renew and differentiate into a wide variety of cell types, making them an appealing option for disease- and regenerative medicine therapies. They have been used to model human disease and have a huge potential for use in drug discovery and cellular therapy. In particular, iPSCs generated from diseased cells can be used as a tool for studying the disease mechanisms and for potential therapies.

This breakthrough discovery has created a powerful new way to “de-differentiate” cells whose developmental fates had been previously assumed to be determined. In addition, tissues derived from iPSCs will be a nearly identical match to the cell donor and thus probably avoid rejection by the immune system.

Several classes of vectors have been used to induce pluripotency when overexpressing the requisite combination of stemness genes. Viral vectors are currently used as main delivery system to introduce the reprogramming factors into adult cells. However, this process has to be carefully controlled and tested before the technique can lead to useful treatment for humans.

In view of these considerations, there is a huge felt need of developing new or alternative methods allowing somatic adult cells reprogramming in order to induce and to obtain a large amount stem-like cells showing high plasticity to be used for cell transplantation and therefore to cure diseases. In particular, the diseases of interest are monogenic genetic diseases such as for example hemophilia.

### SUMMARY

The method of the present invention solves the technical problem reported above by inducing pluripotent stem cells from CD34⁺ cells, fibroblasts or mononuclear cells (MNC). Indeed, these cells when undergo to the present method acquire embryonic stem cell-like phenotype, showing the morphology of these cells, similar gene expression pattern and epigenetic profile, and high plasticity.

The method here disclosed can be applied to obtain iPSCs from adult cells isolated from healthy or diseased individuals. In particular, the applicant found that adult hemophilic cells can be reprogrammed for obtaining iPSCs that can be genetically corrected for the mutated gene involved in the pathogenesis of hemophilia, in particular FVIII, and then they can be administered after being differentiated into endothelial cells to rescue the hemophilic phenotype.

The method for inducing pluripotent stem cells or embryonic stem-like cells of the present invention comprises the following steps:
(i) Having differentiated and/or somatic cells said cells selected from: fibroblasts, lymphocytes, mononuclear cells, and CD34+ cells said cells being isolated from an individual;
(ii) Reprogramming said cells by transducing the cells with a viral vector comprising a DNA sequence codifying at least one transcription factor selected from: Oct4, Sox-2, Klf4 and c-Myc, more preferably the combination of Oct-4, Sox-2 and Klf-4, and/or at least one small RNA molecule, preferably selected from: mi RNA 302 and/or 367;
(iii) Culturing said reprogrammed cells in a medium specific for stem cells to isolate stable reprogrammed cell clones characterized by not more than 4 copies of the viral vector, said viral vector being preferably removable (excisable), preferably by using a PLox/Cre strategy.

In this context, being excisable means that the expression cassette that was inserted can be removed by the use of enzymes. One of the method to excise the inserted DNA involves using PLox/Cre system, a site-specific recombination system. The enzyme Cre recombinase, originally derived from the P1 bacteriophage, recognizes specific 34 base-pair DNA sequences called lox sites. Lox sites are added to an expression cassette so that they flank a sequence of DNA that can be removed, that is well known to a skilled man in this field.

In particular, the mononuclear cells express at least one of the following markers: CD3, CD11 b, CD14, and CD19; while the CD34+ cells are isolated from blood, preferably from peripheral and/or cord blood and/or bone marrow.

Moreover, according to a preferred embodiment, the individual is a healthy individual or a diseased individual, preferably affected by a genetic disease, preferably hemophilia, more preferably type A hemophilia.

Therefore, according to a further embodiment of the present invention, the cells isolated from the diseased individual, are genetically corrected, preferably by gene transfer or gene therapy, for example when the individual is affected by hemophilia A, by transducing into the diseased cells, preferably by using a viral vector, FVIII gene or its variants as here disclosed, or any further gene involved in the coagulation cascade. Alternatively the cells isolated from the diseased individual, are genetically corrected by using TALENs or Crisp/Cas strategy well known to the skilled man in this field. Preferably, FVIII or its variants, or said any further gene involved in the coagulation cascade is under the expression control of Vascular Endothelial Cadherin promoter or its variants; or FVIII promoter or its variants.

According to a preferred embodiment, the cells are activated before step (ii). This activation step is performed by culturing the cells at least 48-70 hours till 4-10 days in a serum-free and/or xeno-free medium comprising cytokines preferably selected from: IL-3, IL-6, IL-7, stem cell factor (SCF), GM-CSF, thrombopoietin (TPO) and FLT3-ligand (FLT3L).

The concentration of said cytokines ranges preferably from 20 ng/ml to 100 ng/ml. The viral vector used for the transduction is preferably a lentiviral or retroviral vector and the transducing step is performed:
(i) By at least one inoculation of the viral vector at a multiplicity of infection (MOI) ranging from 5 to 100, preferably from 5 to 50, more preferably from 5 to 10, still more preferably from 5 to 7; and/or
(ii) On a cell amount ranging from 50.000 to 500.000, preferably from 100.000 to 300.000, more preferably from 150.000 to 250.000; and/or
(iii) The viral vector has a titer ranging from 10⁸ TU/ml to 10¹⁰ TU/ml, preferably from 5*10⁸ TU/ml to 8*10⁹ TU/ml, more preferably from 8*10⁸ TU/ml to 5*10⁹ TU/ml; and/or
(iv) In a volume ranging from 50 μl to 500 μl, preferably from 100 μl to 300 μl, more preferably 150 μl to 200 μl.

According to a preferred embodiment, before step (iii) the cells are cultured for at least 48-72 hours in a serum free medium specific for stem cells comprising a pre-mixed cocktail of recombinant human cytokines preferably: IL3, IL7, IL6, GM-CSF and combination thereof; and/or SCF, FLT3-ligand, TPO.

The step (iii) is performed preferably on a feeder layer, preferably a fibroblast feeder layer. Moreover, this step lasts for fibroblasts preferably at least 6 weeks, more preferably from 6 to 12 weeks; for CD34+ preferably at least 6 weeks, more preferably from 2 to 8 weeks.

A further aspect of the present invention refers to induced pluripotent stem cells or embryonic-like cells obtained/obtainable according to the method of the invention that are characterized by:
Embryonic stem cell-like morphology and therefore they are compact with defined borders; and/or
Positive at alkaline phosphatase staining; and/or
Expressed stem cell nuclear and surface antigens, preferably selected from the group consisting of: Oct4, Sox2, Klf4, Tra1-81 and Ssea-3/4; and/or
Unmethylated state of NANOG promoter; and/or
increase in telomeres therefore reactivation of telomerase complex; and/or
A normal karyotype; and/or
The ability to differentiate all the cell types derived from the three germ layers A further aspect of the present invention refers to a method for differentiating induced pluripotent stem cells or any embryonic stem like cells into endothelial cells, wherein said method comprises the following steps:
(i) Inducing the formation of embryo bodies starting from the induced pluripotent stem cells or embryonic-like cells preferably by:
(ia) Plating the cells in a medium specific for embryo bodies at a concentration ranging from 5 to 50, preferably from 10 to 30, more preferably about 20 colonies/plate to obtain the formation of embryo bodies; and/or
(ib) after about 48 hours from step (ia), the obtained embryo bodies are cultured in suspension by using a medium specific for embryo bodies further comprising BMP4 at a concentration ranging from 5 to 40 mg/ml, preferably from 10 to 30 mg/ml, more preferably 15 to 25 mg/ml, more preferably about 20 mg/ml; and/or
(ic) after about 90-100 hours from step (ia) further adding to the medium specific for embryo bodies FGF at a concentration ranging from 5 to 40, preferably from 10 to 30, more preferably from 15 to 25, more preferably about 20 ng/ml; and/or
(id) after about 130-150 hours from step (ia) the obtained embryo bodies are seeded on a gelatin coated plate in a medium specific for embryo bodies comprising: FGF at a concentration ranging from 5 to 40 ng/ml, preferably from 10 to 30 ng/ml, more preferably from 15 to 25 ng/ml, more preferably about 20 ng/ml; and/or VEGF at a concentration ranging from 30 to 70 ng/ml, preferably from 40-60 ng/ml, more preferably about 50 ng/ml; and/or
(ie) after about 180-200 hours from step (ia) the medium specific for embryo bodies is replaced by a medium including VEGF at a concentration 30-70 ng/ml, preferably 40-60 ng/ml, more preferably about 50 ng/ml until the end of culturing 20 days; and/or
(ii) Collecting the cultured cells wherein said cells are endothelial cells. According to a preferred embodiment of the invention, after 7-15 days, preferably about 10 days from step (ib) the cells attached, preferably to the gelatin coating, preferably in an amount ranging from 80.000 to 150.000 cells, preferably about 100.000 cells, are transduced with a lentiviral vector preferably carrying small RNA molecules, preferably miRNAs, more preferably miRNA126 sequence, preferably SEQ ID NO: 19, and/or miRNA let7b, preferably SEQ ID NO: 20. Preferably, the miRNA is expressed under the control of a promoter, preferably the spleen focus forming virus (SFFV) promoter. Eventually the miRNAs, preferably miRNA126 and miRNA let7b, preferably SEQ ID NO: 19 and 20, are cotransduced. Preferably, the transduction is performed by using a multiplicity of infection (MOI) ranging from 5 to 100, preferably from 5 to 50, more preferably from 10.

A further aspect of the present invention refers to endothelial cells obtained by the method disclosed above.

A further aspect of the present invention refers to a pharmaceutical composition comprising the induced pluripotent stem cells and/or the (differentiated) endothelial cells obtained by the present methods, preferably corrected for the genetic disease, preferably type A hemophilia, and at least one further pharmaceutical acceptable agents, such as carriers, diluents, adjuvants, growth factors. Preferably, the composition further comprises small molecules and/or endothelial specific transcription factors such as Ets1, Ets2 and ERG and/or miRNAs, preferably miRNA126 and/or miRNAlet7b. A further aspect of the present invention refers to induced pluripotent stem cells, or the (differentiated) endothelial cells, or the pharmaceutical composition for use as a medicament, preferably for cell therapy, more preferably for treating a disease, preferably a genetic disease, more preferably type A hemophilia.

### BRIEF DESCRIPTION OF THE FIGURES

A more complete understanding of the present invention can be obtained by considering the following figures that refer to the subsequent detailed description and examples.

FIG. 1 shows embryonic stem cells-like morphology of MNC-derived healthy and hemophilic iPSCs (A, C respectively) and the positivity to alkaline phosphatase staining (B, D) one of the pluripotency markers.

FIG. 2 shows immunofluorescence staining for nuclear (Oct4, Sox2) and surface (Ssea3) pluripotency markers on MNC-derived healthy and hemophilic iPSCs (A, B respectively).

FIG. 3 shows that MNC-iPSCs-derived endothelial cells (MNC ECs) acquired a cobblestone-like morphology (A, B) and expressed endothelial specific markers (KDR, FVIII, CD31, VEC) (C).

FIG. 4 shows immunofluorescence staining for the endothelial markers WVF and FVIII on healthy MNC ECs.

FIG. 5 shows iPSCs generated from cord blood CD34+ cells. iPSCs were positive at alkaline phosphatase staining (A). iPSCs expressed endogenous stem cells factors (B) but not the exogenous (C). Immunofluorescence confirmed the expression of stem cells markers like Oct4, Sox2, SSea-4 and Tra 1-81 (D).

FIG. 6 shows iPSCs generated from peripheral blood CD34+ cells. Both healthy and hemophilic were positive at alkaline phosphatase staining (A, B respectively), and expressed endogenous stem cells factors (Oct4, Sox2, and Klf4) (C, D) but not the exogenous (E, F).

FIG. 7 shows the endothelial markers (CD105, Tie2, vWF, KDR, CD31 and VEC) expression at RNA (A) and protein (B) levels by the hemophilic iPSCs-derived ECs both non-corrected and corrected for FVIII expression.

FIG. 8 shows that healthy, non-corrected and corrected iPSCs-derived ECs efficiently acquired endothelial capability to form tubules when plated on matrigel.

### DEFINITIONS

In the contest of the present invention, “differentiated cells” mean preferably mammalian adult mature differentiated cells and/or mammalian somatic cells. In other words, the differentiated cells of the invention are any biological cell forming the body of an organism other than a gamete, germ cell, gametocyte or undifferentiated stem cell. Preferably, these cells are fibroblasts, CD34+ cells, lymphocytes or mononuclear cells. In particular, CD34+ cells mean hematopoietic progenitor cells found in cord and/or peripheral blood isolated from the mononuclear cells. In this context, said cells are particularly isolated by centrifuging on Ficoll gradient followed by positive immunomagnetic sorting.

In the contest of the present invention, “reprogramming” means the process of inducing adult mature differentiated cells to come back to an embryonic-like pluripotent state, preferably by erasing and remodeling of epigenetic marks, such as DNA methylation, during mammalian development. Reprogramming can be induced artificially, for example, by introducing exogenous factors, usually transcription factors, into the cells to be reprogrammed. In this context, reprogramming often refers to the generation of induced pluripotent stem cells from mature cells such as adult fibroblasts, CD34+ cells, lymphocytes or mononuclear cells. This allows the production of stem cells for biomedical research, such as research into stem cell therapies, without the use of embryos. It is carried out by the transfection of stem-cell associated genes into mature cells preferably using viral vectors, preferably lentiviral vectors.

In the contest of the present invention, “multiplicity of infection (MOI)” means the number of vector particles transducing each cell in culture.

In the contest of the present invention, “viral vector titration” means the method of determining the amount of viral vector particles with an unknown concentration. In the contest of the present invention, “transduction” means the process of a vector particle entrance into a cell, and the integration of the DNA sequences introduced into the vector in the genome after reverse transcription.

In the contest of the present invention, “transcription factor” means one or a set of proteins that bind a specific DNA sequence to initiate and regulate the transcription of a gene or genes responsible for the acquisition and maintenance of the pluripotency (stemness genes). Therefore, in this context transcription factor is also synonymous of stemness gene or sequence. Examples of transcription factors used for the reprogramming are the following: Octamer binding protein 3 or 4 (Oct3/4), sex-determining region Y-box2 (Sox2), homeobox protein Nanog, the Krüppel like factor 4 (Klf4), proto-oncogene c-Myc, and Lin-homolog 28 (Lin28).

In the context of the present invention, “cell potency” means the cell's ability to differentiate into other cell types. The more cell types a cell can differentiate into, the greater is its potency. Potency is also described as the gene activation potential within a cell, which like a continuum begins with totipotency to designate a cell with the most differentiation potential, pluripotency, multipotency, oligopotency and finally unipotency. In the contest of the present invention, “pluripotency” means a stem cell that has the potential to differentiate into any of the three germ layers: endoderm (interior stomach lining, gastrointestinal tract, lungs), mesoderm (muscle, bone, blood, urogenital), or ectoderm (epidermal tissues and nervous system). However, cell pluripotency is a continuum, ranging from the completely pluripotent cell that can form every cell of the embryo proper, e.g., embryonic stem cells and iPSCs, to the incompletely or partially pluripotent cell that can form cells of all three germ layers but that may not exhibit all the characteristics of completely pluripotent cells.

In the contest of the present invention, “induced pluripotent stem cells (iPSCs)” mean pluripotent embryonic stem cell-like cells obtained from adult cells preferably through the introduction of transcription factors necessary to acquire and maintain the defining properties of embryonic stem cells. Indeed, these cells show for example self-renewal capability and a huge plasticity, meaning that they can differentiate into almost all cell types. However, these cells do not pose any ethical concern since they are obtained from adult/somatic cells and not from the embryo.

In the contest of the present invention, “embryonic stem-like cells” mean cells generated from adult cells showing morphology, gene expression pattern, epigenetic profile, self-renewal and pluripotency capability as much as the embryonic stem cells.

### DETAILED DESCRIPTION OF THE INVENTION

The present invention refers to a method for inducing pluripotent stem cells or embryonic stem-like cells comprising the following steps:
(i) Having differentiated and/or somatic cells isolated from an individual said cells being preferably selected from: fibroblasts, lymphocytes, mononuclear cells, and CD34+ cells, preferably from peripheral or cord blood.
(ii) Reprogramming said cells by inducing in said cells the expression of at least one stemness gene (transcription factor) and/or sequence, preferably selected from Oct4, Sox-2, Klf4 and c-Myc, more preferably the combination of Oct-4, Sox-2 and Klf-4, and/or at least one small RNA molecule, preferably a miRNA, more preferably selected from: miRNA 302 and/or 367.

The sequences of the stemness genes and/or of the small RNA molecules preferably used in the present invention are listed in Table I. Oct 4 is preferably SEQ ID NO: 1, Sox-2 is preferably SEQ ID NO: 2, Klf4 is preferably SEQ ID NO: 3, miRNA302 is preferably SEQ ID NO: 4 and/or 5; miRNA 367 is preferably SEQ ID NO: 6.

The method of the present invention allows to obtain induced pluripotent stem cells (iPSCs) or embryonic stem-like cells starting from differentiated/somatic cells. In other words, by applying the method of the present invention, differentiated or somatic cells are dedifferentiated or transdifferentiated or reprogrammed into cells showing a stem cell-like phenotype. This means also that somatic or differentiated cells, undergoing to the present method, acquire staminal phenotype instead of being destined to the death. Therefore, the method of the present invention can be also named a method for reprogramming somatic or differentiated cells into pluripotent stem cells or pluripotent stem-like cells.

The fibroblasts are preferably obtained from a biopsy. Preferably, said fibroblasts are isolated after the enzymatic digestion of biopsy.

Mononuclear cells (MNCs) are preferably isolated from blood, more preferably from peripheral blood. The isolation is performed by using the common techniques know for the scope, such as by centrifuging on Ficoll gradient. MNCs preferably express at least one or combinations of the following markers: CD3, CD11b, CD14, and CD19.

CD34⁺ cells are preferably isolated from blood, more preferably from peripheral and/or cord blood and/or bone marrow. In a still more preferred embodiment CD34⁺ cells are isolated from MNCs.

According to a preferred embodiment the isolation of CD34⁺ are performed by sorting, preferably using beads, preferably magnetic beads.

Preferably, the differentiated/somatic cells are isolated from an adult individual. Said individual can be a healthy individual or a diseased individual. Preferably, the diseased individual is affected by a genetic, disease, preferably monogenic genetic disease, preferably hemophilia, more preferably type A hemophilia. Therefore, in case of isolation from individuals affected by type A hemophilia, the differentiated/somatic cells are named hemophilia A cells or HA differentiated/somatic cells.

Preferably, the differentiated/somatic cells isolated from a diseased individual are corrected for the genetic defect (the mutation) causing the disease before being reprogrammed or after being reprogrammed.

In particular, HA differentiated/somatic cells, preferably HA MNCs, CD34+ cells or fibroblasts are corrected before, more preferably after being reprogrammed. The correction is preferably performed by gene therapy, preferably transducing a viral, preferably lentiviral (LV), vector comprising FVIII gene or its variants or any further gene involved in the coagulation cascade. Preferably FVIII is B-Deleted Domain (BDD)—FVIII, more preferably SEQ ID NO:7. In other words, FVIII gene or its variants and/or any further gene of the coagulation is/are expressed in the HA somatic cells to rescue the hemophilic phenotype.

The gene expression used for the correction of the disease, preferably FVIII expression, is preferably controlled by an endothelial specific promoter, meaning that the gene to be expressed is introduced into a vector under the control of an endothelial specific promoter. The promoter is preferably the VEC promoter or DNA sequences derived from VEC promoter, more preferably SEQ ID NO: 8; and/or FVIII promoter or DNA sequences derived from FVIII promoter, more preferably selected from SEQ ID NO: 9-18.

All the sequences disclosed in the patent application are listed in Table I. Any sequence showing at least 80% of identity with the sequences here disclosed and listed in Table I has to be considered part of the present invention.

TABLE I

| Sequence | Name | Number |
|---|---|---|
| atggccggccacctggccagcgatttcgccttcagccctccacctggcggaggcg | Oct4 | SEQ ID NO 1 |
| gagatggacctggcggccctgaacctggctgggtggaccctcggacctggctga | | |
| gctttcagggccctccaggcggacctggaattggccctggcgtgggccctggatct | | |
| gaagtgtggggcatccctccctgccccccaccctacgagttttgcggcggcatggc | | |
| ctactgtggccctcaggtcggagtgggactggtgcctcagggcggcctggaaacc | | |
| tctcagcctgagggcgaagccggcgtcggcgtggagagcaactctgacggagc | | |
| cagccctgagccttgtaccgtgacccctggcgccgtgaagctggaaaaagagaa | | |
| gctggaacagaaccccgaggaaagccaggacatcaaggccctgcagaaaga | | |
| actggaacagttcgccaagctgctgaagcagaagcggatcacactgggatacac | | |
| ccaggccgatgtgggcctgaccctgggcgtgctgttcggcaaggtgttcagccag | | |
| accaccatctgcagattcgaagccctgcagctgagcttcaagaacatgtgcaagc | | |
| tgcggcccctgctgcagaaatgggtggaggaagccgacaacaacgagaacctg | | |
| caggaaatctgcaaggccgagacactggtgcaggcccggaagcggaagcgga | | |
| ccagcatcgagaacagagtgcggggcaacctggaaaacctgttcctgcagtgcc | | |
| ccaagcccaccctgcagcagatcagccacattgctcagcagctcggcctggaaa | | |
| aggacgtcgtgagagtgtggttctgcaaccggcggcagaagggcaagcggagc | | |
| agcagcgactacgcccagagagaggacttcgaggccgctggcagcccttttagc | | |
| ggcggacccgtgtcctttcctctggcccctggccctcactttggcacccctggctacg | | |
| gcagcccacacttcaccgccctgtacagcagcgtgccctttccagagggcgagg | | |
| ccttcccccctgtgtccgtgaccaccctgggcagccccatgca | | |
| gcatgtacaacatgatggaaaccgagctgaagcctcccggccctcagcagaca | Sox-2 | SEQ ID NO 2 |
| agtggaggcggcggaggcaattctacagccgccgctgccggcggaaaccaga | | |
| agaacagccccgacagagtgaagcggcccatgaacgccttcatggtctggtcca | | |
| gaggacagaggcggaagatggcccaggaaaaccccaagatgcacaacagcg | | |
| agatcagcaagagactgggcgccgagtggaagctgctgagcgagacagagaa | | |
| gcggcccttcatcgacgaggccaagcggctgagagccctgcacatgaaggaac | | |
| accccgactacaagtaccggcccagaagaaagaccaagaccctgatgaagaa | | |
| ggacaagtacaccctgccaggcggactgctggccccaggcggcaattctatggc | | |
| cagcggagtgggagtgggagctggactgggagccggcgtgaaccagcggatg | | |
| gacagctacgcccacatgaacggctggtccaacggcagctacagcatgatgca | | |
| ggaccagctgggctaccctcagcaccctggcctgaatgcccatggcgccgctca | | |
| gatgcagcccatgcaccgctacgatgtgtccgccctgcagtacaacagcatgacc | | |
| agcagccagacctacatgaatggcagccccacctacagcatgtcctacagccag | | |
| cagggcacaccaggcatggccctgggctctatgggcagcgtggtcaagagcga | | |
| ggccagcagcagccctcctgtggtcaccagcagctcccacagcagagccccttgt | | |
| caggccggcgacctgcgggacatgatcagcatgtacctgcctggcgccgaagtg | | |
| cctgaacctgccgcccctagcagactgcacatgagccagcactaccagagcgg | | |
| ccctgtgcctggcaccgccatcaatggcaccctgcccct | | |
| tctggcatggccgtgagcgacgccctgctgcccagcttcagcacctttgccagcgg | KLF4 | SEQ ID NO 3 |
| acctgcaggcagagagaaaaccctgcggcaggctggcgcccctaacaaccggt | | |
| ggcgggaggaactgtcccacatgaagagactgccccccgtgctgcccggcaga | | |
| ccttatgatctggccgctgccaccgtggccaccgatctggaatctggcggagccgg | | |
| cgctgcctgtggcggaagcaacctggcccccctgcccagacgggagacagaag | | |
| agttcaacgacctgctggacctggacttcatcctgagcaacagcctgacccaccct | | |
| cctgagtctgtggccgccaccgtgtctagcagcgccagcgccagcagctctagct | | |
| ctcctagctcttctggccctgccagcgcccccagcacctgtagcttcacctaccccat | | |
| cagagccggcaatgatcctggcgtggcaccaggcggaacaggcggaggactc | | |
| ctgtacggcagagagtctgccccaccccccaccgcccccttcaacctggccgac | | |
| atcaacgacgtgtcccccagcggaggctttgtggccgagctgctgaggcctgagc | | |
| tggaccccgtgtacatccccccacagcagcctcagcctccaggcggcggactgat | | |
| gggcaagttcgtgctgaaagccagcctgagcgcccctggctctgagtatggctccc | | |
| ccagcgtgatcagcgtgtccaagggcagccctgatggctctcaccctgtggtggtg | | |
| gccccttacaatggcggccctcccagaacctgccccaagatcaagcaggaagc | | |
| cgtcagcagctgtacacacctgggcgctggccctccactgagcaacggacacag | | |
| gcctgccgcccacgactttccactgggcagacagctccctagcagaaccacccc | | |
| caccctggggctggaagaggtgctgagcagcagagactgccaccctgccctgcc | | |
| tctgccacctggctttcatcctcacccaggccccaactaccccagcttcctgcccga | | |
| ccagatgcagcctcaggtgccccccctgcactaccaggaactgatgcccccagg | | |
| cagctgcatgcccgaggaacccaagcccaagcggggcagaagaagctggccc | | |
| cggaagagaaccgccacccacacctgtgattacgccggctgcggcaagaccta | | |
| caccaagagcagccacctgaaggcccacctgagaacccacaccggcgagaa | | |
| gccctaccactgcgactgggacggctgcggctggaagttcgccagaagcgacg | | |
| agctgacccggcactacagaaagcacaccggccaccggcccttccagtgccag | | |
| aagtgcgaccgggccttcagcagatccgaccacctggctctgcatatgaagcggc | | |
| actttt | | |
| ccaccacttaaacgtggatgtacttgctttgaaactaaagaagtaagtgcttccatgt | miRNA302A | SEQ ID NO 4 |
| tttggtgatgg | | |
| cctctactttaacatggaggcacttgctgtgacatgacaaaaataagtgcttccatgtt | miRNA302D | SEQ ID NO 5 |
| tgagtgtgg | | |
| ccattactgttgctaatatgcaactctgttgaatataaattggaattgcactttagcaat | miRNA367 | SEQ ID NO 6 |
| ggtgatgg | | |
| atgcaaatagagctctccacctgcttctttctgtgccttttgcgattctgctttagtgccac | BDD-FVIII | SEQ ID NO: 7 |
| cagaagatactacctgggtgcagtggaactgtcatgggactatatgcaaagtgatc | | |
| tcggtgagctgcctgtggacgcaagatttcctcctagagtgccaaaatcttttccattc | | |
| aacacctcagtcgtgtacaaaaagactctgtttgtagaattcacggatcaccttttca | | |
| acatcgctaagccaaggccaccctggatgggtctgctaggtcctaccatccaggct | | |
| gaggtttatgatacagtggtcattacacttaagaacatggcttcccatcctgtcagtctt | | |
| catgctgttggtgtatcctactggaaagcttctgagggagctgaatatgatgatcaga | | |
| ccagtcaaagggagaaagaagatgataaagtcttccctggtggaagccatacat | | |
| atgtctggcaggtcctgaaagagaatggtccaatggcctctgacccactgtgcctta | | |
| cctactcatatctttctcatgtggacctggtaaaagacttgaattcaggcctcattgga | | |
| gccctactagtatgtagagaagggagtctggccaaggaaaagacacagaccttg | | |
| cacaaatttatactactttttgctgtatttgatgaagggaaaagttggcactcagaaac | | |
| aaagaactccttgatgcaggatagggatgctgcatctgctcgggcctggcctaaaa | | |
| tgcacacagtcaatggttatgtaaacaggtctctgccaggtctgattggatgccaca | | |
| ggaaatcagtctattggcatgtgattggaatgggcaccactcctgaagtgcactca | | |
| atattcctcgaaggtcacacatttcttgtgaggaaccatcgccaggcgtccttggaa | | |
| atctcgccaataactttccttactgctcaaacactcttgatggaccttggacagtttcta | | |
| ctgttttgtcatatctcttcccaccaacatgatggcatggaagcttatgtcaaagtaga | | |
| cagctgtccagaggaaccccaactacgaatgaaaaataatgaagaagcggaa | | |
| gactatgatgatgatcttactgattctgaaatggatgtggtcaggtttgatgatgacaa | | |
| ctctccttcctttatccaaattcgctcagttgccaagaagcatcctaaaacttgggtac | | |
| attacattgctgctgaagaggaggactgggactatgctcccttagtcctcgcccccg | | |
| atgacagaagttataaaagtcaatatttgaacaatggccctcagcggattggtagg | | |
| aagtacaaaaaagtccgatttatggcatacacagatgaaacctttaagactcgtga | | |
| agctattcagcatgaatcaggaatcttgggacctttactttatggggaagttggagac | | |
| acactgttgattatatttaagaatcaagcaagcagaccatataacatctaccctcac | | |
| ggaatcactgatgtccgtcctttgtattcaaggagattaccaaaaggtgtaaaacatt | | |
| tgaaggattttccaattctgccaggagaaatattcaaatataaatggacagtgactgt | | |
| agaagatgggccaactaaatcagatcctcggtgcctgacccgctattactctagttt | | |
| cgttaatatggagagagatctagcttcaggactcattggccctctcctcatctgctac | | |
| aaagaatctgtagatcaaagaggaaaccagataatgtcagacaagaggaatgt | | |
| catcctgttttctgtatttgatgagaaccgaagctggtacctcacagagaatatacaa | | |
| cgctttctccccaatccagctggagtgcagcttgaggatccagagttccaagcctcc | | |
| aacatcatgcacagcatcaatggctatgtttttgatagtttgcagttgtcagtttgtttgc | | |
| atgaggtggcatactggtacattctaagcattggagcacagactgacttcctttctgtc | | |
| ttcttctctggatataccttcaaacacaaaatggtctatgaagacacactcaccctatt | | |
| cccattctcaggagaaactgtcttcatgtcgatggaaaacccaggtctatggattctg | | |
| gggtgccacaactcagactttcggaacagaggcatgaccgccttactgaaggtttc | | |
| tagttgtgacaagaacactggtgattattacgaggacagttatgaagatatttcagc | | |
| atacttgctgagtaaaaacaatgccattgaaccaagaagcttctcccaaaaccca | | |
| ccagtcttgaaacgccatcaacgggaaataactcgtactactcttcagtcagatca | | |
| agaggaaattgactatgatgataccatatcagttgaaatgaagaaggaagattttg | | |
| acatttatgatgaggatgaaaatcagagcccccgcagctttcaaaagaaaacac | | |
| gacactattttattgctgcagtggagaggctctgggattatgggatgagtagctcccc | | |
| acatgttctaagaaacagggctcagagtggcagtgtccctcagttcaagaaagttg | | |
| ttttccaggaatttactgatggctcctttactcagcccttataccgtggagaactaaatg | | |
| aacatttgggactcctggggccatatataagagcagaagttgaagataatatcatg | | |
| gtaactttcagaaatcaggcctctcgtccctattccttctattctagccttatttcttatga | | |
| ggaagatcagaggcaaggagcagaacctagaaaaaactttgtcaagcctaatg | | |
| aaaccaaaacttacttttggaaagtgcaacatcatatggcacccactaaagatga | | |
| gtttgactgcaaagcctgggcttatttctctgatgttgacctggaaaaagatgtgcact | | |
| caggcctgattggaccccttctggtctgccacactaacacactgaaccctgctcatg | | |
| ggagacaagtgacagtacaggaatttgctctgtttttcaccatctttgatgagaccaa | | |
| aagctggtacttcactgaaaatatggaaagaaactgcagggctccctgcaatatcc | | |
| agatggaagatcccacttttaaagagaattatcgcttccatgcaatcaatggctaca | | |
| taatggatacactacctggcttagtaatggctcaggatcaaaggattcgatggtatct | | |
| gctcagcatgggcagcaatgaaaacatccattctattcatttcagtggacatgtgttc | | |
| accgtacgaaaaaaagaggagtataaaatggcactgtacaatctctatccaggtg | | |
| tttttgagacagtggaaatgttaccatccaaagctggaatttggcgggtggaatgcct | | |
| tattggcgagcatctacatgctgggatgagcacactttttctggtgtacagcaataag | | |
| tgtcagactcccctgggaatggcttctggacacattagagattttcagattacagcttc | | |
| aggacaatatggacagtgggccccaaagctggccagacttcattattccggatca | | |
| atcaatgcctggagcaccaaggagcccttttcttggatcaaggtggatctgttggca | | |
| ccaatgattattcacggcatcaagacccagggtgcccgtcagaagttctccagcct | | |
| ctacatctctcagtttatcatcatgtatagtcttgatgggaagaagtggcagacttatc | | |
| gaggaaattccactggaaccttaatggtcttctttggcaatgtggattcatctgggata | | |
| aaacacaatatttttaaccctccaattattgctcgatacatccgtttgcacccaactcat | | |
| tatagcattcgcagcactcttcgcatggagttgatgggctgtgatttaaatagttgcag | | |
| catgccattgggaatggagagtaaagcaatatcagatgcacagattactgcttcat | | |
| cctactttaccaatatgtttgccacctggtctccttcaaaagctcgacttcacctccaa | | |
| gggaggagtaatgcctggagacctcaggtgaataatccaaaagagtggctgcaa | | |
| gtggacttccagaagacaatgaaagtcacaggagtaactactcagggagtaaaa | | |
| tctctgcttaccagcatgtatgtgaaggagttcctcatctccagcagtcaagatggcc | | |
| atcagtggactctcttttttcagaatggcaaagtaaaggtttttcagggaaatcaaga | | |
| ctccttcacacctgtggtgaactctctagacccaccgttactgactcgctaccttcga | | |
| attcacccccagagttgggtgcaccagattgccctgaggatggaggttctgggctg | | |
| cgaggcacaggacctctactga | | |
| ctagtagcagaaacaaggtcctctggaagagcaactgatgctcttaggtactgaa | VEC | SEQ ID NO: 8 |
| gcatcatcctgccccagagaccactcgcatatgaagcacacatattcagtctgcctt | promoter | |
| acttgtgttaatgattgccagtgtccctctgacctcctagccctgaaaaggtgtggcct | | |
| gaaggtcatttcagagacggggagagctgctcagagaagccaatcggcgagtct | | |
| aggacacacagacaggatctagtcccagagttcgctagcctaggtgagcgtccc | | |
| ctggccccttataccacttccttctccagcttgcatctaattcgctctggcagaccatcg | | |
| tgtttcctgtcttcctggcagcctccagcacgctcagtgctactccctcgcatgcgccc | | |
| tcctcccagtaccttctctgactccagtgggcttggagtgcgaggaggaagggtga | | |
| ggaaggggtgaaatcaggtattggatccacagggggtctgaagagcactagcct | | |
| ggccttttgggactgaacttctgctatgaagacctccactgccatccctggagtccgg | | |
| ggcacatccaaggnttgctgtccatcgtttaactgtttacagatgacaacaatgactc | | |
| gtgttcggggcagaaatatcaccagggctagagtacaaaaggagtttgcattgatg | | |
| gccggacaggcctgtccctggaccagcctgcgacgctgagtatgagacccagcg | | |
| gaagtgctaccctggcagacgtgtcactgagtacacagaccaccaaggcaggc | | |
| agctctcggggaagctgtctatgctgggccagcccaccttgagggcagggaaca | | |
| gaacagattgtggcagagaggaaaatgtggagcttctgtttgttcacagacacacg | | |
| cactcgcccacgcacgcacgcacgcacgcacgcacgcacgaatgcacgcacg | | |
| cagtagttgaatgctatggattccgctcagagctgagaacagccccagcgacagtt | | |
| ccctggcctctctccttactctgatgtcctcatctgtcttcacatggtctcaggacgcta | | |
| atactccatcctaatgtacactcctttccctgggcctccgttccagttcagttctcagag | | |
| gacctggagggagtgattggctacaccaactttgctttcgttcaccaagcccatgtct | | |
| ctacttgggtgtctaatgggcatctccaacattacctaccccaaacagaaaacccttt | | |
| cttccccccaaccacaccccaccctacccccacagtattttctccatgcccggaaa | | |
| gatctgctctcttatggtccctctttgcctcactgaaaagcaggacaagttggggaac | | |
| ttcccaaacttttatgcatgaagaaacccaggcaatttgccaaaaggtacactctgg | | |
| gggtctgtcatttactctgagccagaaccctgaaatttttactaacccatcacataatg | | |
| aatgaagagaatctttttctttttttttttttttctttttttttggtttttcgagacagggtttctctgt | | |
| atagccctggctatcctggaacacactctgtagaccaggctggcctcgaactcaga | | |
| aatccacctgcctctgcctcccgagtgctgggattaaaggcgtgcgccaccacgc | | |
| ctggctgaatgaagagaatcttgacctcatctccccagcctcttggtcctgagggac | | |
| cctggtctacctactgctttgctgtcttcttagctcttcttacttttttgctgactcagacctat | | |
| ggctatctccattatacagatgaggagactgaggcatggatccctggttggtccatg | | |
| gtcacgtgaagcccatcacccagtatttgtaaagtgagatgggccaggctggtacc | | |
| ttggaactgaaactcacactgccctacctggaagaatctgacaggcaaaatctgct | | |
| gctgaaagtgattgtctgtcacgtttctcagctgcccgactctgagaactccacagcc | | |
| ccctttcgttccaccatactacagagtcgccacggaaagccggctctgtggagaag | | |
| ctgaggtagctgggtttctgtctgggttactctgtccagcgaggaaacaagtacctta | | |
| gacccactaagcctctgctttctgaactgtaaagtgggggatatgacacctgcctcc | | |
| cagggatggctgaatgctctggcagaagcttagagcccccacagctacccctag | | |
| gctcacagctcctccgatgagacctagaattgaggtatgagttgaataccccaggc | | |
| aggtccaaggcttccacgggcccaggctgaccaagctgaggccgcccaccgta | | |
| gggcttgcctatctgcaggcagctcacaaaggaacaataacaggaaaccatccc | | |
| gaggggaagtgggccagggcaagttggaaaacctgcctccctcccagcctgggt | | |
| gtggctcccctctcccctcctgaggcaatcaactgtgctctccacaaagctcggccc | | |
| tggacagactcgactagaggatccaccggtcgccacca | | |
| gagctcaccatggctacattctgatgtaaagagatatatcctatacctgggccaaat | LV.pF8.1 | SEQ ID NO: 9 |
| gtaaacagcctggaaaagtgttaggttaaaaacaaaacaaaataaataaatgaa | | |
| taaatgccaggtggttatgagtgctattgagaaaaatgaagccaagagggatatc | | |
| agtgatgcaggtgggggtaaagagcttacaacataaatgtggtgttccatatttaaa | | |
| cctcattcaacagggaagattggagctgaaatgtgaaggagttgtgggagtggaa | | |
| ctacgtggaaatctgggggaaaggtgttttgggtaaaagaaatagcaagtgttgag | | |
| gtccaggggcatgagtgtgcttgatattttagggaagagtaaggagaccagtataa | | |
| ccagagtgagatgagactacagaggtcaggagaaagggcatgcagaccatgtg | | |
| ggatgctctaggacctaggccatggtaaagatgtagggttttaccctgatggaggtc | | |
| agaagccattggaggattctgagaagaggagtgacaggactcgctttatagtttta | | |
| aattataactataaattatagtttttaaaacaatagttgcctaacctcatgttatatgtaa | | |
| aactacagttttaaaaactataaattcctcatactggcagcagtgtgaggggcaag | | |
| ggcaaaagcagagagactaacaggttgctggttactcttgctagtgcaagtgaatt | | |
| ctagaatcttcgacaacatccagaacttctcttgctgctgccactcaggaagagggt | | |
| tggagtaggctaggaataggagcacaaattaaagctcctgttcactttgacttctcc | | |
| atccctctcctcctttccttaaaggttctgattaaagcagacttatgcccctactgctctc | | |
| agaagtgaatgggttaagtttagcagcctcccttttgctacttcagttcttcctgtggctg | | |
| cttcccactgataaaaaggaagcaatcctatcggttactgcttagtgctgagcacat | | |
| ccagtgggtaaagttccttaaaatgctctgcaaagaaattgggacttttcattaaatc | | |
| agaaattttacttttttcccctcctgggagctaaagatattttagagaagaattaaccttt | | |
| tgcttctccagttgaacatttgtagcaataagtc | | |
| gtttttaaaacaatagttgcctaacctcatgttatatgtaaaactacagttttaaaaact | LV.pF8.2 | SEQ ID NO: 10 |
| ataaattcctcatactggcagcagtgtgaggggcaagggcaaaagcagagaga | | |
| ctaacaggttgctggttactcttgctagtgcaagtgaattctagaatcttcgacaacat | | |
| ccagaacttctcttgctgctgccactcaggaagagggttggagtaggctaggaata | | |
| ggagcacaaattaaagctcctgttcactttgacttctccatccctctcctcctttccttaa | | |
| aggttctgattaaagcagacttatgcccctactgctctcagaagtgaatgggttaagt | | |
| ttagcagcctcccttttgctacttcagttcttcctgtggctgcttcccactgataaaaagg | | |
| aagcaatcctatcggttactgcttagtgctgagcacatccagtgggtaaagttcctta | | |
| aaatgctctgcaaagaaattgggacttttcattaaatcagaaattttacttttttcccctc | | |
| ctgggagctaaagatattttagagaagaattaaccttttgcttctccagttgaacatttg | | |
| tagcaataagtc | | |
| Ggggctcgctcgctcagtacctggaggcgagttcctgacgcgactgcgactcaat | LV.pF8.3 | SEQ ID NO: 11 |
| cctcgcctggtgaagaatattttacctatgactcactgaaaataaagacggctgagt | | |
| gaccgtgtttgttcatgtaaacattgaacaaatatttatcggcttctgcgatgtgtccta | | |
| ctcttttagtggaggaagacacattttatttatgtatttaatttttcttttgaattttacatgcg | | |
| agttatacttaataaaactcacttcaaaatataccttcaacagaaaatccagcaaca | | |
| gtttctattatgttagttaaaacagccagtcttttcctttacttttaaaaattattcataaatg | | |
| taattagtgaatgataataaacattgacatctgatccactgctttaggagtgacacaa | | |
| atgaagttaactcaggctattttctttataatcattgtgctattgttttctttttcttttcaattat | | |
| actgcttaatataggattttgtggcaccataggagttgaGGagctcaccatggctac | | |
| attctgatgtaaagagatatatcctatacctgggccaaatgtaaacagcctggaaa | | |
| agtgttaggttaaaaacaaaacaaaataaataaatgaataaatgccaggtggttat | | |
| gagtgctattgagaaaaatgaagccaagagggatatcagtgatgcaggtggggg | | |
| taaagagcttacaacataaatgtggtgttccatatttaaacctcattcaacagggaa | | |
| gattggagctgaaatgtgaaggagttgtgggagtggaactacgtggaaatctggg | | |
| ggaaaggtgttttgggtaaaagaaatagcaagtgttgaggtccaggggcatgagt | | |
| gtgcttgatattttagggaagagtaaggagaccagtataaccagagtgagatgag | | |
| actacagaggtcaggagaaagggcatgcagaccatgtgggatgctctaggacct | | |
| aggccatggtaaagatgtagggttttaccctgatggaggtcagaagccattggagg | | |
| attctgagaagaggagtgacaggactcgctttatagttttaaattataactataaatta | | |
| tagtttttaaaacaatagttgcctaacctcatgttatatgtaaaactacagttttaaaaa | | |
| ctataaattcctcatactggcagcagtgtgaggggcaagggcaaaagcagagag | | |
| actaacaggttgctggttactcttgctagtgcaagtgaattctagaatcttcgacaac | | |
| atccagaacttctcttgctgctgccactcaggaagagggttggagtaggctaggaa | | |
| taggagcacaaattaaagctcctgttcactttgacttctccatccctctcctcctttcctt | | |
| aaaggttctgattaaagcagacttatgcccctactgctctcagaagtgaatgggtta | | |
| agtttagcagcctcccttttgctacttcagttcttcctgtggctgcttcccactgataaaa | | |
| aggaagcaatcctatcggttactgcttagtgctgagcacatccagtgggtaaagttc | | |
| cttaaaatgctctgcaaagaaattgggacttttcattaaatcagaaattttacttttttcc | | |
| cctcctgggagctaaagatattttagagaagaattaaccttttgcttctccagttgaac | | |
| atttgtagcaataagtc | | |
| Ggggctcgctcgctcagtacctggaggcgagttcctgacgcgactgcgactcaat | LV.pF8.4 | SEQ ID NO: 12 |
| cctcgcctggtgaagaatattttacctatgactcactgaaaataaagacggctgagt | | |
| gaccgtgtttgttcatgtaaacattgaacaaatatttatcggcttctgcgatgtgtccta | | |
| ctcttttagtggaggaagacacattttatttatgtatttaatttttcttttgaattttacatgcg | | |
| agttatacttaataaaactcacttcaaaatataccttcaacagaaaatccagcaaca | | |
| gtttctattatgttagttaaaacagccagtcttttcctttacttttaaaaattattcataaatg | | |
| taattagtgaatgataataaacattgacatctgatccactgctttaggagtgacacaa | | |
| atgaagttaactcaggctattttctttataatcattgtgctattgttttctttttcttttcaattat | | |
| actgcttaatataggattttgtggcaccataggagttgaGGtttttaaaacaatagttg | | |
| cctaacctcatgttatatgtaaaactacagttttaaaaactataaattcctcatactggc | | |
| agcagtgtgaggggcaagggcaaaagcagagagactaacaggttgctggttact | | |
| cttgctagtgcaagtgaattctagaatcttcgacaacatccagaacttctcttgctgct | | |
| gccactcaggaagagggttggagtaggctaggaataggagcacaaattaaagct | | |
| cctgttcactttgacttctccatccctctcctcctttccttaaaggttctgattaaagcaga | | |
| cttatgcccctactgctctcagaagtgaatgggttaagtttagcagcctcccttttgcta | | |
| cttcagttcttcctgtggctgcttcccactgataaaaaggaagcaatcctatcggttac | | |
| tgcttagtgctgagcacatccagtgggtaaagttccttaaaatgctctgcaaagaaa | | |
| ttgggacttttcattaaatcagaaattttacttttttcccctcctgggagctaaagatatttt | | |
| agagaagaattaaccttttgcttctccagttgaacatttgtagcaataagtc | | |
| Tcgccaccacttggcttccggcacgtggggcagatgtttccattcccacggcggca | LV.pF8.5 | SEQ ID NO: 13 |
| gcggaagagggagggccgggcgcgccgcggctgcttgcagtctccgcaagcg | | |
| gctacatcacagagctcagcgtgcggtgtcacaggccccgcggtcccgcccaac | | |
| agatgcaccgagatgcgcgtgcgcagaaagcgtcccgggggtgaggctccctc | | |
| cctcgctctccctctactcccgccccactctcccccactttcccccctccacccaccg | | |
| cggccgtcggggctcgctcgctcagtacctggaggcgagttcctgacgcgactgc | | |
| gactcaatcctcgcctggtgaagaatattttacctatgactcactgaaaataaagac | | |
| ggctgagtgaccgtgtttgttcatgtaaacattgaacaaatatttatcggcttctgcgat | | |
| gtgtcctactcttttagtggaggaagacacattttatttatgtatttaatttttcttttgaatttt | | |
| acatgcgagttatacttaataaaactcacttcaaaatataccttcaacagaaaatcc | | |
| agcaacagtttctattatgttagttaaaacagccagtcttttcctttacttttaaaaattatt | | |
| cataaatgtaattagtgaatgataataaacattgacatctgatccactgctttaggagt | | |
| gacacaaatgaagttaactcaggctattttctttataatcattgtgctattgttttctttttctt | | |
| ttcaattatactgcttaatataggattttgtggcaccataggagttgaGGagctcacc | | |
| atggctacattctgatgtaaagagatatatcctatacctgggccaaatgtaaacagc | | |
| ctggaaaagtgttaggttaaaaacaaaacaaaataaataaatgaataaatgcca | | |
| ggtggttatgagtgctattgagaaaaatgaagccaagagggatatcagtgatgca | | |
| ggtgggggtaaagagcttacaacataaatgtggtgttccatatttaaacctcattcaa | | |
| cagggaagattggagctgaaatgtgaaggagttgtgggagtggaactacgtgga | | |
| aatctgggggaaaggtgttttgggtaaaagaaatagcaagtgttgaggtccaggg | | |
| gcatgagtgtgcttgatattttagggaagagtaaggagaccagtataaccagagtg | | |
| agatgagactacagaggtcaggagaaagggcatgcagaccatgtgggatgctct | | |
| aggacctaggccatggtaaagatgtagggttttaccctgatggaggtcagaagcc | | |
| attggaggattctgagaagaggagtgacaggactcgctttatagttttaaattataac | | |
| tataaattatagtttttaaaacaatagttgcctaacctcatgttatatgtaaaactacagt | | |
| tttaaaaactataaattcctcatactggcagcagtgtgaggggcaagggcaaaag | | |
| cagagagactaacaggttgctggttactcttgctagtgcaagtgaattctagaatctt | | |
| cgacaacatccagaacttctcttgctgctgccactcaggaagagggttggagtagg | | |
| ctaggaataggagcacaaattaaagctcctgttcactttgacttctccatccctctcct | | |
| cctttccttaaaggttctgattaaagcagacttatgcccctactgctctcagaagtgaa | | |
| tgggttaagtttagcagcctcccttttgctacttcagttcttcctgtggctgcttcccactg | | |
| ataaaaaggaagcaatcctatcggttactgcttagtgctgagcacatccagtgggt | | |
| aaagttccttaaaatgctctgcaaagaaattgggacttttcattaaatcagaaatttta | | |
| cttttttcccctcctgggagctaaagatattttagagaagaattaaccttttgcttctcca | | |
| gttgaacatttgtagcaataagtc | | |
| Tcgccaccacttggcttccggcacgtggggcagatgtttccattcccacggcggca | LV.pF8.6 | SEQ ID NO: 14 |
| gcggaagagggagggccgggcgcgccgcggctgcttgcagtctccgcaagcg | | |
| gctacatcacagagctcagcgtgcggtgtcacaggccccgcggtcccgcccaac | | |
| agatgcaccgagatgcgcgtgcgcagaaagcgtcccgggggtgaggctccctc | | |
| cctcgctctccctctactcccgccccactctcccccactttcccccctccacccaccg | | |
| cggccgtcggggctcgctcgctcagtacctggaggcgagttcctgacgcgactgc | | |
| gactcaatcctcgcctggtgaagaatattttacctatgactcactgaaaataaagac | | |
| ggctgagtgaccgtgtttgttcatgtaaacattgaacaaatatttatcggcttctgcgat | | |
| gtgtcctactcttttagtggaggaagacacattttatttatgtatttaatttttcttttgaatttt | | |
| acatgcgagttatacttaataaaactcacttcaaaatataccttcaacagaaaatcc | | |
| agcaacagtttctattatgttagttaaaacagccagtcttttcctttacttttaaaaattatt | | |
| cataaatgtaattagtgaatgataataaacattgacatctgatccactgctttaggagt | | |
| gacacaaatgaagttaactcaggctattttctttataatcattgtgctattgttttctttttctt | | |
| ttcaattatactgcttaatataggattttgtggcaccataggagttgaGGtttttaaaac | | |
| aatagttgcctaacctcatgttatatgtaaaactacagttttaaaaactataaattcctc | | |
| atactggcagcagtgtgaggggcaagggcaaaagcagagagactaacaggttg | | |
| ctggttactcttgctagtgcaagtgaattctagaatcttcgacaacatccagaacttct | | |
| cttgctgctgccactcaggaagagggttggagtaggctaggaataggagcacaa | | |
| attaaagctcctgttcactttgacttctccatccctctcctcctttccttaaaggttctgatt | | |
| aaagcagacttatgcccctactgctctcagaagtgaatgggttaagtttagcagcct | | |
| cccttttgctacttcagttcttcctgtggctgcttcccactgataaaaaggaagcaatcc | | |
| tatcggttactgcttagtgctgagcacatccagtgggtaaagttccttaaaatgctctg | | |
| caaagaaattgggacttttcattaaatcagaaattttacttttttcccctcctgggagct | | |
| aaagatattttagagaagaattaaccttttgcttctccagttgaacatttgtagcaataa | | |
| gtc | | |
| cagcagttcccacaaacgttaccctcacaatgaatccagccatttttcaccctctcca | 0 to 2350 5′ | SEQ ID NO: 15 |
| gtggtaccatcatagcccaagccgccaccatttctcacccccggttaacaggccac | FVIII | |
| cctccttctacccttatcctgctagagtttgttttatctacagtgatcagaaagatcagc | promoter | |
| ctaaaagataattctgatcaccaccctcctctactcacaacccggccgtgtctcccc | sequence | |
| attgccctcagtgtagaagtcaatgtccctttgctgaaatgcaaccttagtgaaacttt | | |
| ccatgactaacctcctttaaaattgcaacctggtccacccttactcccccttacccca | | |
| cttctcttttttgcacagcacttattttaccttctaacatactgtataatgtactcatgtattgt | | |
| aattattgcttatcatccctctttcagttgcttatatttttcatcaatgtgtacccagtgccta | | |
| ggacaatatctgtctaggacaaatgggtagttatgtggctgtaggcaagccatttaa | | |
| cctctctgtacctcagttactttatctgtatccactttgcggtgttgtcatgaggattaaat | | |
| cagatagcctatgtgtagcacctggcagtgaatttatcaccctgtactgtaactgtcta | | |
| cttttctgtctcctccattggactgtcattcccagggggttgggaactgggatttcttcatt | | |
| tctgaggcatagaagtatagcatagtggttaggagcatgacttctggagccagagt | | |
| acatgggtttgaatgctaccactcacaagctgtgtggccatggagaagttgcctaac | | |
| ctctccgtgcttcagtttcatcacccataaaatgaaggtaagaatagtacctgtattta | | |
| aaagcacctagaacagttcctggcatatagtgtcagctgtcatctctgcatccttgta | | |
| cctgtcagagaggagtgtttatcaaaggggcttcttgctgcctgtttccaaaccagtc | | |
| gacaatataccaattgctccctaacacattcttgtttgtgcagaactgagctcaatgat | | |
| aacatttttatagcaaccctgatcaagtttcttctcataatctcttacactttgaggcccc | | |
| tgcaggggccctcactctccctaataaacattaacctgagtagggtgtttgagctca | | |
| ccatggctacattctgatgtaaagagatatatcctatacctgggccaaatgtaaaca | | |
| gcctggaaaagtgttaggttaaaaacaaaacaaaataaataaatgaataaatgc | | |
| caggtggttatgagtgctattgagaaaaatgaagccaagagggatatcagtgatg | | |
| caggtgggggtaaagagcttacaacataaatgtggtgttccatatttaaacctcattc | | |
| aacagggaagattggagctgaaatgtgaaggagttgtgggagtggaactacgtg | | |
| gaaatctgggggaaaggtgttttgggtaaaagaaatagcaagtgttgaggtccag | | |
| gggcatgagtgtgcttgatattttagggaagagtaaggagaccagtataaccaga | | |
| gtgagatgagactacagaggtcaggagaaagggcatgcagaccatgtgggatg | | |
| ctctaggacctaggccatggtaaagatgtagggttttaccctgatggaggtcagaa | | |
| gccattggaggattctgagaagaggagtgacaggactcgctttatagttttaaattat | | |
| aactataaattatagtttttaaaacaatagttgcctaacctcatgttatatgtaaaacta | | |
| cagttttaaaaactataaattcctcatactggcagcagtgtgaggggcaagggcaa | | |
| aagcagagagactaacaggttgctggttactcttgctagtgcaagtgaattctagaa | | |
| tcttcgacaacatccagaacttctcttgctgctgccactcaggaagagggttggagt | | |
| aggctaggaataggagcacaaattaaagctcctgttcactttgacttctccatccctc | | |
| tcctcctttccttaaaggttctgattaaagcagacttatgcccctactgctctcagaagt | | |
| gaatgggttaagtttagcagcctcccttttgctacttcagttcttcctgtggctgcttccc | | |
| actgataaaaaggaagcaatcctatcggttactgcttagtgctgagcacatccagt | | |
| gggtaaagttccttaaaatgctctgcaaagaaattgggacttttcattaaatcagaaa | | |
| ttttacttttttcccctcctgggagctaaagatattttagagaagaattaaccttttgcttct | | |
| ccagttgaacatttgtagcaataagtc | | |
| Ggggctcgctcgctcagtacctggaggcgagttcctgacgcgactgcgactcaat | Enhancer | SEQ ID NO: 16 |
| cctcgcctggtgaagaatattttacctatgactcactgaaaataaagacggctgagt | Short | |
| gaccgtgtttgttcatgtaaacattgaacaaatatttatcggcttctgcgatgtgtccta | | |
| ctcttttagtggaggaagacacattttatttatgtatttaatttttcttttgaattttacatgcg | | |
| agttatacttaataaaactcacttcaaaatataccttcaacagaaaatccagcaaca | | |
| gtttctattatgttagttaaaacagccagtcttttcctttacttttaaaaattattcataaatg | | |
| taattagtgaatgataataaacattgacatctgatccactgctttaggagtgacacaa | | |
| atgaagttaactcaggctattttctttataatcattgtgctattgttttctttttcttttcaattat | | |
| actgcttaatataggattttgtggcaccataggagttgag | | |
| Tcgccaccacttggcttccggcacgtggggcagatgtttccattcccacggcggca | Enhancer | SEQ ID NO: 17 |
| gcggaagagggagggccgggcgcgccgcggctgcttgcagtctccgcaagcg | Long | |
| gctacatcacagagctcagcgtgcggtgtcacaggccccgcggtcccgcccaac | | |
| agatgcaccgagatgcgcgtgcgcagaaagcgtcccgggggtgaggctccctc | | |
| cctcgctctccctctactcccgccccactctcccccactttcccccctccacccaccg | | |
| cggccgtcggggctcgctcgctcagtacctggaggcgagttcctgacgcgactgc | | |
| gactcaatcctcgcctggtgaagaatattttacctatgactcactgaaaataaagac | | |
| ggctgagtgaccgtgtttgttcatgtaaacattgaacaaatatttatcggcttctgcgat | | |
| gtgtcctactcttttagtggaggaagacacattttatttatgtatttaatttttcttttgaatttt | | |
| acatgcgagttatacttaataaaactcacttcaaaatataccttcaacagaaaatcc | | |
| agcaacagtttctattatgttagttaaaacagccagtcttttcctttacttttaaaaattatt | | |
| cataaatgtaattagtgaatgataataaacattgacatctgatccactgctttaggagt | | |
| gacacaaatgaagttaactcaggctattttctttataatcattgtgctattgttttctttttctt | | |
| ttcaattatactgcttaatataggattttgtggcaccataggagttgag | | |
| tcgccaccacttggcttccggcacgtggggcagatgtttccattcccacggcggca | Full 5′ FVIII | SEQ ID NO: 18 |
| gcggaagagggagggccgggcgcgccgcggctgcttgcagtctccgcaagcg | promoter | |
| gctacatcacagagctcagcgtgcggtgtcacaggccccgcggtcccgcccaac | sequence | |
| agatgcaccgagatgcgcgtgcgcagaaagcgtcccgggggtgaggctccctc | | |
| cctcgctctccctctactcccgccccactctcccccactttcccccctccacccaccg | | |
| cggccgtcggggctcgctcgctcagtacctggaggcgagttcctgacgcgactgc | | |
| gactcaatcctcgcctggtgaagaatattttacctatgactcactgaaaataaagac | | |
| ggctgagtgaccgtgtttgttcatgtaaacattgaacaaatatttatcggcttctgcgat | | |
| gtgtcctactcttttagtggaggaagacacattttatttatgtatttaatttttcttttgaatttt | | |
| acatgcgagttatacttaataaaactcacttcaaaatataccttcaacagaaaatcc | | |
| agcaacagtttctattatgttagttaaaacagccagtcttttcctttacttttaaaaattatt | | |
| cataaatgtaattagtgaatgataataaacattgacatctgatccactgctttaggagt | | |
| gacacaaatgaagttaactcaggctattttctttataatcattgtgctattgttttctttttctt | | |
| ttcaattatactgcttaatataggattttgtggcaccataggagttgagtaaaaataaa | | |
| aggaataaaaatataccttatctggccgggcgcggtggctcacgcctgtaatttcag | | |
| cagtttcggaggccgaggcgggcggatcacgcggtcaggagatcgaggccatc | | |
| ctggctaacatggtgaaaccccgtctctactaaaaatacaaaaaattagccgggc | | |
| atggtggcggccgcctgtagtcccagctactcgggaggctgaggcaggagaatg | | |
| gcgtgaacccgggaggcggagcttgcagtgagccgagatcgcgacactgcact | | |
| ccagcctgggcgacagagtgagactgcgtctccaaaaaaaaaagaaaaaata | | |
| cgttatctatgaagatttccaatttgatttctatttatcacaaatggccacagtactccttt | | |
| gtactttaccacataccatattgtattcagtaattatttgtgaatatgtaattgataatatt | | |
| gtaggttttagagaatccttgaaaacatgaaaatttggtaatggggtctattttgattatt | | |
| tatttatttatttatttattttatttttgagacagagtctcgctcttgttgcccaggctggagtg | | |
| cagtggcgcgatctcggctcactgcaagctccacctcccgggttcaagcgattctc | | |
| ctgcctcagcctcccaagtagctgggactacaggcacgtgccaccatgcccggct | | |
| aattttttgtatttttagtagaggaggagtttcatcttgttagctaggatggtctagatctc | | |
| ctgacctcgtgatctgcccgcctcagcctcccaaagtgctgggattacaggtgtgag | | |
| ccaccgtgcccggccatattttgatttaaaatttagcaataatagataaaattttcaat | | |
| caactaagcccttgggccagggaatgctattccttaaaaagtgcttctatcaatatag | | |
| cctctgactcattactttgttaatttttaaattgtatttcattcctgattaacattcccaccca | | |
| gattattaattatacaatctgttaactgtagaacctcaaacatgttggattgtactgtattt | | |
| gtctggaagacacatttttaaaacattgtaatcgctataagagaagcactgggaaa | | |
| gaaaggagcttctatgcctgcagtgcctgaggagccctttaacagtgtgccccgcc | | |
| cctaagctactcatgcagtcatccccatcccagttagtcaactttattccaaaaaactt | | |
| ggtgttccaaatttttccttctcaaagcccacagatccaaaattcatcagcagttccca | | |
| caaacgttaccctcacaatgaatccagccatttttcaccctctccagtggtaccatca | | |
| tagcccaagccgccaccatttctcacccccggttaacaggccaccctccttctaccc | | |
| ttatcctgctagagtttgttttatctacagtgatcagaaagatcagcctaaaagataatt | | |
| ctgatcaccaccctcctctactcacaacccggccgtgtctccccattgccctcagtgt | | |
| agaagtcaatgtccctttgctgaaatgcaaccttagtgaaactttccatgactaacct | | |
| cctttaaaattgcaacctggtccacccttactcccccttaccccacttctcttttttgcac | | |
| agcacttattttaccttctaacatactgtataatgtactcatgtattgtaattattgcttatc | | |
| atccctctttcagttgcttatatttttcatcaatgtgtacccagtgcctaggacaatatctg | | |
| tctaggacaaatgggtagttatgtggctgtaggcaagccatttaacctctctgtacctc | | |
| agttactttatctgtatccactttgcggtgttgtcatgaggattaaatcagatagcctatg | | |
| tgtagcacctggcagtgaatttatcaccctgtactgtaactgtctacttttctgtctcctcc | | |
| attggactgtcattcccagggggttgggaactgggatttcttcatttctgaggcataga | | |
| agtatagcatagtggttaggagcatgacttctggagccagagtacatgggtttgaat | | |
| gctaccactcacaagctgtgtggccatggagaagttgcctaacctctccgtgcttca | | |
| gtttcatcacccataaaatgaaggtaagaatagtacctgtatttaaaagcacctaga | | |
| acagttcctggcatatagtgtcagctgtcatctctgcatccttgtacctgtcagagagg | | |
| agtgtttatcaaaggggcttcttgctgcctgtttccaaaccagtcgacaatataccaat | | |
| tgctccctaacacattcttgtttgtgcagaactgagctcaatgataacatttttatagca | | |
| accctgatcaagtttcttctcataatctcttacactttgaggcccctgcaggggccctc | | |
| actctccctaataaacattaacctgagtagggtgtttgagctcaccatggctacattct | | |
| gatgtaaagagatatatcctatacctgggccaaatgtaaacagcctggaaaagtgt | | |
| taggttaaaaacaaaacaaaataaataaatgaataaatgccaggtggttatgagt | | |
| gctattgagaaaaatgaagccaagagggatatcagtgatgcaggtgggggtaaa | | |
| gagcttacaacataaatgtggtgttccatatttaaacctcattcaacagggaagattg | | |
| gagctgaaatgtgaaggagttgtgggagtggaactacgtggaaatctgggggaa | | |
| aggtgttttgggtaaaagaaatagcaagtgttgaggtccaggggcatgagtgtgctt | | |
| gatattttagggaagagtaaggagaccagtataaccagagtgagatgagactac | | |
| agaggtcaggagaaagggcatgcagaccatgtgggatgctctaggacctaggc | | |
| catggtaaagatgtagggttttaccctgatggaggtcagaagccattggaggattct | | |
| gagaagaggagtgacaggactcgctttatagttttaaattataactataaattatagtt | | |
| tttaaaacaatagttgcctaacctcatgttatatgtaaaactacagttttaaaaactata | | |
| aattcctcatactggcagcagtgtgaggggcaagggcaaaagcagagagacta | | |
| acaggttgctggttactcttgctagtgcaagtgaattctagaatcttcgacaacatcca | | |
| gaacttctcttgctgctgccactcaggaagagggttggagtaggctaggaatagga | | |
| gcacaaattaaagctcctgttcactttgacttctccatccctctcctcctttccttaaagg | | |
| ttctgattaaagcagacttatgcccctactgctctcagaagtgaatgggttaagtttag | | |
| cagcctcccttttgctacttcagttcttcctgtggctgcttcccactgataaaaaggaag | | |
| caatcctatcggttactgcttagtgctgagcacatccagtgggtaaagttccttaaaat | | |
| gctctgcaaagaaattgggacttttcattaaatcagaaattttacttttttcccctcctgg | | |
| gagctaaagatattttagagaagaattaaccttttgcttctccagttgaacatttgtagc | | |
| aataagtc | | |
| ctgaggaccgccaggcaggggctggtgctgggcggggggcggcgggccctcc | miRNA let7b | SEQ ID NO: 19 |
| cgcagtgcaaggccgggcctggcggggtgaggtagtaggttgtgtggtttcaggg | | |
| cagtgatgttgcccctcggaagataactatacaacctactgccttccctgaggagcc | | |
| cagtgacacgaccccatgggagggccgccccctacctcagtgacacgacccca | | |
| cgggagggctgccccccacctcagtgacctgcagggggcctagccgaagctgg | | |
| gtgggcatctgggagctagattcaataaagctgttctgaccatgaacttggaactgg | | |
| cccc | | |
| gacggtacactctgtgtgcccaagggagggccccccagggtggcccccaaccc | miRNA 126 | SEQ ID NO: 20 |
| gacaggtaaacagccctggctgtgcctggcctggggaggcgggcaggcagtgg | | |
| acattgccgtgtggctgttaggcatggtggggggcactggaatctgggcggaagg | | |
| cggtggggactccctctccagggagggaggatggggagggaggataggtgggtt | | |
| cccgagaactgggggcaggttgcccggagcctcatatcagccaagaaggcaga | | |
| agtgccccgtcccggggtcctgtctgcatccagcgcagcattctggaagacgcca | | |
| cgcctccgctggcgacgggacattattacttttggtacgcgctgtgacacttcaaact | | |
| cgtaccgtgagtaataatgcgccgtccacggcaccgcatcgaaagcgccgctga | | |
| gacctcagccttgacctccctcagcgtggccgggaccctgagcctctgcgcagag | | |
| ccacccgccccgacgtacttaggcggcatagccctgagacctctggccagcgcc | | |
| aggcaggcagcgggggcggcagaggcctgggcctgagtcttctggctctgcctct | | |
| ccctggggacaggagggagcctgggggtgtgggtggggagccggccggccgt | | |
| gacccagcgcctggctctgcccgcaggagtggacagtgcaatgaaggaagaag | | |
| tgcagaggctgca | | |

Before being reprogrammed, differentiated/somatic cells are preferably cultured or expanded. In other words, they are seeded at a concentration of preferably about 10⁶ cells/ml in a cell culture well containing a defined medium to be expanded (amplified in the number) in vitro.

Preferably, the expansion phase of the cells lasts for at least 48-70 hours till 4-10 days. Preferably for at least 4-5 days.

Preferably, the expansion/culture/growth medium is a chemically defined, serum-free and/or xeno-free medium developed to support, preferably with the addition of appropriate cytokines, the proliferation of the isolated cells. Example of such a medium are: Hematopoietic Growth Medium (HPGM), aMEM, IMDM, StemSpam, or CellGro. The cytokines are preferably of human origin, more preferably selected from: IL-3, IL-6, IL-7, stem cell factor (SCF), GM-CSF, thrombopoietin (TPO) and FLT3-ligand (FLT3L). Preferably, the cytokines are replaced in the medium every 24-72 hours, more preferably every 2 days.

Preferably, the cytokines are used as a mixture. Preferably, a mixture of IL3, IL7, IL6 and GM-CSF for MNCs, or a mixture of SCF, FLT3-ligand, TPO and IL3 for CD34+. The concentration of said cytokines ranges from 20 ng/ml to 100 ng/ml. More preferably, the concentration of the mixture of cytokines ranges from 5 to 25 ng/ml, preferably about 10 ng/ml for MNCs or preferably about 50 ng/ml for CD34+ cells.

The culturing/expansion phase allows cell activation, meaning that cells become more responsive to any external stimuli, preferably more responsive to viral, preferably lentiviral, transduction.

Preferably, when the differentiated/somatic cells are fibroblasts the activation is not required. Therefore, the culturing/expansion phase are not performed.

The expression of the at least one transcription factor (stemness gene or sequence thereof) is induced preferably by using a viral vector (in this case the induction is called transduction), more preferably a retroviral or lentiviral (LV) vector. The sequence codifying the at least one transcription factor and/or the at least one small RNA molecule is introduced into the vector. Preferably, when the transcription factor to be induced is more the one their sequences or portions thereof are inserted in the same vector as a polycistronic construct, in other words the vector is polycystronic. The DNA sequence used for the induction codifies the full length or portion thereof of said transcription factor (stemness gene).

The transduction is preferably performed by at least one inoculation of the viral vector, preferably at a multiplicity of infection (MOI) ranging from 5 to 100, more preferably from 5 to 50, still more preferably from 5 to 10, still more preferably from 5 to 7. Preferably, the cells are transduced in a quantity ranging from 50.000 to 500.000, preferably from 100.000 to 300.000, more preferably from 150.000 to 250.000. The vector has preferably high titration, more preferably ranging from 10⁸ TU/ml to 10¹⁰ TU/ml, still more preferably from 5*10⁸ TU/ml to 8*10⁹ TU/ml, still more preferably from 8*10⁸ TU/ml to 5*10⁹ TU/ml.

The transduction is preferably performed in a small volume, preferably ranging from 50 μl to 500 μl, more preferably from 100 μl to 300 μl, still more preferably 150 μl to 200 μl. After the induction of the transcription factors (transduction) the treated cells (transduced cells) are cultured for at least 48-72 hours in a medium specific for stem cells culturing. Preferably the medium is serum free and generally comprises a pre-mixed cocktail of recombinant human cytokines. Examples of such a medium are: αMEM, Hematopoietic Growth Medium (HPGM), HES, CellGro or StemSpam medium with the specific cytokines. The cytokines are preferably selected from: IL3, IL7, IL6, GM-CSF and combination thereof preferably for MNCs, and/or SCF, FLT3-ligand, TPO, IL3 and combination thereof preferably for CD34+.

Preferably, it is advisable to change the culturing medium every day and more preferably to dissociate the cells, preferably by mechanical means.

Preferably this culturing phase can be omitted when the differentiated starting cells are fibroblasts.

Preferably after the culturing phase, or, preferably, for fibroblasts directly after the induction of the at least one transcription factor (transduction), the treated cells are cultured on a feeder layer, preferably a human fibroblast feeder layer or mouse embryonic fibroblasts (MEF). Alternatively, the cells treated can be grown on Geltrex® Matrix Products (Thermo Fisher Scientific) without feeder cells.

In this context, feeder layer means a coating layer of fibroblasts, generally from human foreskin and/or irradiated, supplying the metabolites necessary to the cells they support. Generally, these fibroblasts do not grow or divide anymore because of the pretreatment with gamma irradiation or drugs such as Mitomycin.

Preferably, fibroblasts are cultured on the feeder layer at least 6 weeks, more preferably from 6 to 12 weeks.

Preferably, CD34+ cells are cultured on the feeder layer at least 6 weeks, more preferably from 2 to 8 weeks.

This phase allows the formation of cell clones and the culturing phase is required for stabilizing the obtained clones.

Preferably, only the stabilized clones characterized by less than 4-6, preferably 1-2 copy/copies of the vector, preferably the viral vector is/are selected. Indeed the applicant has surprisingly found that only these types of clones are stable. Instead, clones having 4-6 copies of the vector or more are unstable.

Preferably, the sequences codifying the transcription factor genes contained into the vector used for inducing their expression in the differentiated/somatic cells are preferably comprised between a self-deleting Cre-lox cassette allowing the removal of the transcription factor genes after induction.

At the end of these phases the selected cell clones show embryonic cell like phenotype. Indeed the applicant found that selected cell colonies showed embryonic stem cell-like morphology, meaning that they were compact with defined borders. Further, the selected cell colonies were preferably positive at alkaline phosphatase staining and, more preferably, expressed also stem cell nuclear and surface antigens, preferably selected from the group consisting of: Oct4, Sox2, Klf4, Tra1-81 and Ssea-3. Finally, the selected cell clones further showed preferably unmethylated state of NANOG promoter, and/or an increase in telomeres length demonstrating the reactivation of telomerase complex and/or a normal karyotype.

These cells are induced pluripotent stem cells (iPSCs) that can be differentiated into several cell lineages, potentially into all the cell types derived from the three germ layers, in other words into all the cell type of a human body.

Therefore, a further aspect of the present invention refers to induced pluripotent stem cells or embryonic-like cells obtained/obtainable according to the method disclosed above characterized by:
Embryonic stem cell-like morphology and therefore they are compact with defined borders; and/or
Positive at alkaline phosphatase staining; and/or
Expressed stem cell nuclear and surface antigens, preferably selected from the group consisting of: Oct4, Sox2, Klf4, Tra1-81 and Ssea-3; and/or
Unmethylated state of NANOG promoter; and/or
Increase in telomeres length therefore reactivation of telomerase complex; and/or
A normal karyotype; and/or
The ability to differentiate all the cell types derived from the three germ layers.

In particular, according to a further aspect of the present invention, the induced pluripotent stem cells or embryonic-like cells obtained/obtainable according to the method disclosed above or any induced pluripotent stem cells or any embryonic-like cells can be differentiated into endothelial cells by using the method here below disclosed.

The applicant set up a new and efficient method for differentiating induced pluripotent stem cells or any embryonic stem like cells into endothelial cells, wherein said method comprises the following steps:
(i) Inducing the formation of embryo bodies starting from the induced pluripotent stem cells obtained by the method disclose above or any induced pluripotent stem cells or embryonic-like cells by:
(ia) Plating the cells, preferably on a low adhesion surface, at a concentration ranging preferably from 5 to 50, more preferably from 10 to 30, still more preferably about 20 colonies/plate for inducing (allowing) embryo bodies formation in a medium specific for embryo bodies culturing, preferably EB medium alone and/or HPGM or HES; and/or
(ib) after about 48 h from step (ia), the obtained embryo bodies are cultured in suspension in the medium specific for embryo bodies culturing further comprising BMP4 at a concentration ranging from 5 to 40 mg/ml, preferably from 10 to 30 mg/ml, more preferably 15 to 25 mg/ml, more preferably about 20 mg/ml; and/or
(ic) after about 90-100 hours from step (ia) further adding to the medium FGF at a concentration ranging from 5 to 40, preferably from 10 to 30, more preferably from 15 to 25, more preferably about 20 ng/ml; and/or
(id) after about 130-150 hours from step (ia) the cultured embryo bodies are seeded on a gelatin coated plate in a medium specific for embryo bodies culturing comprising: FGF at a concentration ranging from 5 to 40 ng/ml, preferably from 10 to 30 ng/ml, more preferably from 15 to 25 ng/ml, more preferably about 20 ng/ml; and/or VEGF at a concentration ranging from 30 to 70 ng/ml, more preferably from 40 to 60 ng/ml, more preferably about 50 ng/ml; and/or
(ie) after about 180-200 hours from step (ia) the medium specific for embryo bodies culturing is replaced by a medium including VEGF at a concentration ranging from 30 to 70 ng/ml, preferably from 40 to 60 ng/ml, more preferably about 50 ng/ml until the end of culturing 20 days; and/or
(ii) Collecting the cultured cells.

According to a preferred embodiment of the invention, after 7-15 days, preferably after about 10 days from step (ib) the cells attached, preferably to the gelatin coating, preferably in an amount ranging from 80.000 to 150.000 cells, preferably about 100.000 cells, are transduced with a lentiviral vector preferably carrying small RNA molecules, preferably miRNAs, more preferably miRNA126 sequence, preferably SEQ ID NO: 19, and/or miRNA let7b, preferably SEQ ID NO: 20. Preferably, the miRNA is expressed under the control of a promoter, preferably the spleen focus forming virus (SFFV) promoter. Eventually the miRNAs, preferably miRNA126 and miRNA let7b, preferably SEQ ID NO: 19 and 20, are cotransduced. Preferably, the transduction is performed by using a multiplicity of infection (MOI) ranging from 5 to 100, preferably from 5 to 50, more preferably from 10.

The collected cells are endothelial cells, indeed they express at least one endothelial marker, preferably selected from: Tie2, CD105, vWF, KDR, CD31 and VEC. These cells express again FVIII or its variants when the HA somatic cells used as starting cells are reprogrammed and corrected for the genetic mutation by transducing the cells with a viral vector comprising FVIII gene or its variants, preferably SEQ ID NO: 4, preferably under the control of an endothelial specific promoter, preferably FVIII promoter or its variants, preferably selected from SEQ ID NO: 9-15, or VEC promoter or its variants, preferably SEQ ID NO: 8.

The induced pluripotent stem cells or any embryonic stem-like cells are obtained/obtainable preferably from differentiated/somatic adult cells, preferably from MNCs, CD34+ cells or fibroblasts.

According to a preferred embodiment, these cells derive from a healthy individual or from a patient. Said patient is preferably affected by a genetic disease, preferably a monogenic genetic disease, such as for example hemophilia, preferably type A hemophilia.

Therefore, the induced pluripotent stem cells or any embryonic stem like cells can be preferably HA induced pluripotent stem cells or any embryonic stem like cells if they are not genetically corrected before being differentiated into endothelial cells.

Indeed, in some embodiments the induced pluripotent stem cells or any embryonic stem like cells even if they derived from a patient affected by hemophilia A (from HA differentiated/somatic cells), they can be corrected before being obtained or more preferably after (at the end of) the reprogramming process.

The genetic correction is performed according to the previous disclosed method.

A further aspect of the present invention, is a pharmaceutical composition comprising the induced pluripotent stem cells and/or any embryonic stem like cells obtained/obtainable by the claimed method and/or the (differentiated) endothelial cells, preferably corrected for the genetic disease, such as hemophilia, more preferably type A hemophilia, obtained according to the method here disclosed, and at least one further pharmaceutical acceptable agents, such as carriers, diluents, adjuvants, growth factors and devices, microcarriers beads or hydrogel matrix coupled with transduced cells able to secrete FVIII for phenotypic correction. Preferably, the composition further comprises small molecules and/or endothelial specific transcription factors, preferably Ets1, Ets2 or ERG, and/or miRNAs, preferably miRNA126 and/or miRNAlet7b.

According to a further aspect of the present invention, the induced pluripotent stem cells or any embryonic stem like cells obtained/obtainable by the claimed method and/or the (differentiated) endothelial cells, or the pharmaceutical composition comprising such cells can be used as a medicament, preferably for cell therapy, more preferably for treating a disease. The disease is preferably a genetic disease, such as a monogenic disease, for example hemophilia, preferably type A hemophilia.

In the context of the present invention, “treating a disease” means reducing the severity of the disease, and/or arresting the development of the disease; and/or inhibiting worsening of the disease; and/or limiting or preventing recurrence of the disease; and/or causing regression of the disease; and/or ameliorating the symptoms of the disease; and/or improving survival of patients.

These cells or the composition comprising such cells can be systematically delivered or administered to be targeted to the tissue in need thereof. Alternatively, they can be locally administered, preferably delivered directly at or nearby the site in need of these cells.

According to further embodiments, the cells or the composition are comprised into a delivery system prior to implantation, preferably an artificially engineered tissue, or introduced into a matrix, preferably a pouch or, alternatively, they are bound to microbeads.

Alternatively, the endothelial cells of the present invention or the composition comprising such cells are used for promoting vasculogenesis and/or angiogenesis.

### Example

MNC and CD34+ Cell Purification and Culture.

Mononuclear cells (MNCs) used in this study were obtained from 5 healthy donors and 20 hemophilic patients. CD34+ cells used in this study were obtained from 2 healthy donors, 1 heterozygous control and 4 hemophilic patients. All volunteer donors provided their written informed consent and the Ethics Committees from the Azienda Ospedaliera Universitaria dell'Ospedale della Carità di Novara approved this consent procedure. The donors were not treated with any mobilizing agent and peripheral blood was obtained in EDTA or heparin tubes by venipuncture.

MNCs were purified from peripheral blood (PB) by Ficoll separation. Briefly, 20 ml of PB were diluted (1:3) with phosphate buffered saline. Then diluted PB were stratified on Ficoll in a ratio of 2:1 and centrifuged at 650×g for 20′. MNC ring was harvest, washed with PBS and centrifuged at 350×g for 10′. Cells pellet was recovered and plated in α-MEM with 10 ng/mL each hIL-3, hIL-6, hIL-7, hGM-CSF. Cells were expanded for 4 days and every 2 days 10 ng/ml of cytokines were added.

CD34⁺ cells were isolated from MNC using the MACS® CD34 MicroBead Kit according to manufacturer's protocol. Isolated cells were expanded for 4 days to obtain approximately 300.000 cells in HPGM medium with 1% human serum albumin, 50 ng/mL of hSCF, hFlt3-ligand, hTPO and hIL-3.

Culture and Irradiation of Human Foreskin Fibroblasts

Human foreskin fibroblasts (HFF—ATCC® SCRC-1041™) were used as feeder layer for iPSCs culture. Specifically HFF were cultured in IMDM with 10% of fetal bovine serum (FBS), 2 mM glutamine, 50 U\ml penicillin and 50 μg/ml streptomycin. Before their use as feeder layer they were mitotically inactivated by gamma ray irradiation (25 Gy) and freezed in aliquots of 10\hat{ }6-2*10\hat{ }6 cells/ml of freezing medium (90% FBS and 10% DMSO). The day before iPSCs expansion, irradiated HFF were plated on a 0.1% gelatin coated plates in IMDM.

iPSCs and Embryoid Bodies (EBs) Culture

iPSCs were cultured and characterized using standard techniques. Specifically, iPSCs were cultured at 37° C. with 5% CO2 on irradiated HFFs in HES medium, consisting of KnockOut DMEM supplemented with 20% KnockOut Serum Replacement, 2 mM Glutammine, 50 μM 2-mercaptoethanol, non-essential amino acids, and 10 ng/ml basic fibroblast growth factor (bFGF). HES medium was changed daily. Once a week, iPSCs were detached mechanically and plated onto fresh HFFs in HES medium. Moreover, the iPSCs can be maintained in a defined surface for feeder free culture using vitronectin. Cells can be maintained for many passages without losing the ability to differentiate.

Vector Transduction for Reprogramming and FVIII Correction 5 days after isolation, both healthy and hemophilic MNC were transduced with third generation self-inactivating Cre-exisable polycystronic lentiviral vectors LV-Lox-SFFV-Oct4-Sox2-Klf4 (LV-SFFV-OSK) by two consecutive spinoculation at MOI 5 for each at 300 g for 1 hour. CD34+ cells were transduced with Cre-exisable polycystronic LV carrying miRNA cluster 302\367 followed by OSK cassette (LV-SFFV-miR-302\367-OSK) or the OS cassette (LV-SFFV-miR-302\367-OS) by a single spinoculation at MOI 5 at 300×g for 1 hour. 2 days after transduction cells were seeded on the top of HFF feeder layer in α-MEM or HPGM and 2 days later medium was changed with HES medium. From 20 up to 45 days colonies appeared. iPSCs were maintained on HFF feeder layer in HES medium. Medium was changed every day. Individual iPSCs colonies were passed by mechanical dissociation.

HA MNC were first genetically corrected by transduction at MOI 10 with a LV carrying the human coagulation factor B domain deleted (hBDD)-FVIII under the control of ubiquitous promoter of phosphoglycerate kinase (PGK). Then, we corrected HA MNC- and CD34-derived iPSCs by transduction with a LV carrying the B domain deleted form of FVIII under the control of VEC endothelial specific promoter (LV-VEC-hBDDFVIII), LV-VEC-GFP was used as transduction control.

iPSCs Staining for Pluripotency Markers

For immunofluorescence staining iPSCs were cultured into slide flasks on irradiated HFF in HES medium. Immunofluorescence was performed using standard protocols. Primary antibodies included anti-OCT4, anti-SOX2, anti-TRA1-81 (1:100) and anti-SSEA3 (1:100). Secondary antibodies included Alexa Fluor 488® Goat anti-Rabbit/Rat IgG (1:500) and Alexa Fluor® 546 Goat anti-Mouse IgG (1:500). Nuclei were stained with 4,6-diamidino-2-phenylindole (DAPI; 1:1000).

For Alkaline Phosphatase (AP) staining, iPSCs were fixed and stained using the Alkaline Phosphatase (AP) detection kit according to the manufacturer's protocol.

RNA isolation and RT-PCR

RNA was isolated by Isol-RNA Lysis Reagent. 1 μg of total RNA was reverse-transcribed with RevertAid First Strand cDNA Synthesis Kit and PCRs were performed on cDNA.

All the PCRs were performed with GoTaq® Flexi DNA Polymerase. PCR protocol was as follow: initial denaturation at 95° C. for 5 min followed by 30 cycles (25 cycles for β-actin) of denaturation at 94° C. for 30″, annealing at 50-62° C. for 30-45″, extension at 72° C. for 60″, and final extension at 72° C. for 7 minutes. Primers, annealing temperatures and product sizes are listed in the table. PCR products were resolved in 2% agarose gels.

Vector Integration and Copy Number Analysis in iPSCs

LV-SFFV-OSK and LV-SFFV-miR-302\367-OSK integration in iPSCs was quantified using genomic DNA purified from cells using Relia Prep gDNA Tissue Miniprep System and diluted to 25 ng/mL. Primers used were: Wpre5′-TGGATTCTGCGCGGGACGTC-3′ and

dNEF 5′-GGCTAAGATCTACAGCTGCCTTG-3′,
GAPDH 5′-AACGTGTCAGTGGTGGACCTG-3′
and
5′-AGTGGGTGTCGCTGTTGAAGT-3′.

qPCR for copy number was performed using the GoTaq® qPCR Master Mix using primers previously described. qPCR protocol was: denaturation at 95° C. for 2 min followed by 40 cycles of denaturation at 95° C. for 15″ and annealing/extension at 60° C. for 60″ according to the manufacturer's protocol.

NANOG Promoter Methylation Analysis

Genomic DNA was isolated purified from MNC, CD34+ cells and iPSCs using ReliaPrepgDNA Tissue Miniprep System. Then 1 μg genomic DNA was bisulfite-converted using EpiTect Kit. A total of 150 ng of converted gDNA was used for PCR using primer amplifying 8 CpG-islands in the Nanog promoter (Forward: 5′-TGGTTAGGTTGGTTTTAAATTTTTG-3′; reverse: 5′-ACCCACCCTTATAAATTCTCAATTA-3′). Amplified products were subcloned into pCR2.1 vectors using the Topo TA cloning Kit (Invitrogen). Individual colonies were picked, plasmid DNA was purified using the NucleoSpin® Plasmid, and DNA was sequenced using M13 Rev and M13 (−20) For primers.

iPSCs Karyotype

Chromosomal analysis of iPSCs was carried using standard G banding method in collaboration with Hospital San Luigi Gonzaga, Orbassano, Italy. Briefly, colchicine (10 μg/ml final concentration) was added to iPSCs seeded in slide flasks at 37° C. for one hour. After this time, cells were washed three times with PBS, incubated with trypsin-EDTA solution for 5 minutes, collected in a fresh tube and centrifuged (700 g for 10 minutes). Obtained pellet was resuspended in hypotonic solution (0.075 M potassium chloride) and incubated at 37° C. for 30 minutes. Cells were then pre-fixed with Carnoy-fixative solution (methanol/glacial acetic acid 3:1) and centrifuged at 700 g for 10 minutes. Finally, the supernatant was discarded; pellet resuspended again in Carnoy solution and suspension was dripped on clean slide. After some days, before staining, slides were immersed in 60° C. solution 1×SCC (sodium chloride and sodium citrate) for 30 minutes. Later, after being washed with running water, each slide was stained by 4 ml of dye (Wright's stain 0.06% and buffer pH 6.8, 1:3) for about 10 minutes, rinsed and dried on a plate. Unmounted slides were examined using Nikon Eclipse 1000 light microscopy and photographed with Genicon software. Thirty high-quality G-banded metaphases were selected for each time. The chromosomes were classified according to the International System for Cytogenetic Nomenclature (ISCN).

Telomeres Length Analyses

Telomeres length was assed using qPCR Multiplex on genomic DNA extracted from iPSCs at 5, 10, 15, 20 passages. On endothelial cells genomic DNA was extracted 10, 20, 25 passages post-differentiation. Real-time PCR was used to assess average telomere length ratio as previously described (Zamperone et al., 2013).

Adipogenic, Osteogenic and Chondrogenic Differentiation

EBs were formed, plated on 0.1% gelatin (Sigma-Aldrich) coated plates and cultured in Mesenchymal Stem Cell Adipogenic Differentiation Medium (MSC) or osteogenic medium consisting in α Minimum Essential Medium, FBS 10%, 0.4 mM ascorbic acid, 1 mM β-glicerophosphate, and 10 nM dexamethasone. Media were changed every 3 days. After 14-20 days, cells were washed in PBS, fixed with 4% PAF and stained with Oil Red O (ORO) for adipogenic and with Alizarin Red (ARS) 40 mM pH 4.1 for osteogenic. The presence of lipid vacuoles and the production of calcium deposits was examined in light microscopy (Leica ICC50HD, 200×, 400× magnification).

For chondrogenic differentiation, iPSCs were cultured for 30 days in 15 mL centrifuge tubes in Chondrogenic Medium. The medium was changed every 2/3 days. Cells were then washed, fixed in 4% PAF, included in OCT, and frozen at −80° C. 4 μm sections were cut, stained using the primary goat antibody against collagen II (1:200), and secondary AlexaFluor546 donkey-anti-goat-IgG antibody (1:500) following standard protocol. Nuclei were stained with DAPI (1:1000) and observed at fluorescence microscope.

Endothelial Cell Differentiation

EBs were formed in 35-mm tissue culture dishes (SARSTEDT AG & Co.) and differentiated in endothelial cells using two different protocols, referred to Vascular Endothelium Growth Factor (VEGF) protocol and Bone Morphogenic 4 (BMP4) protocol. For VEGF protocol, EBs were generated and plated on 6-well tissue culture gelatin coated plates (0.1% gelatin) in EB medium with 50 ng/ml of VEGF until the end of differentiation (20 days).

For BMP4 protocol, EBs formation was induced and after 2 days of growth in EB medium alone, BMP4 was added (20 mg/ml) (day 2). At day 4, EBs were cultured in EB medium 20 ng/ml basic FGF was added. At day 6 EBs were plated on 6-well tissue culture 0.1% gelatin coated plates in EB medium with 20 ng/ml of basic FGF (bFGF) and 50 ng/ml of VEGF (R&D Systems). At day 8 medium was changed with EB medium added only by 50 ng/ml of VEGF until the end of differentiation (20 days). Cells were passed when plates were 90% confluent and maintained in EB medium with 50 ng/ml of VEGF.

Endothelial Cell Transduction with miRNAs

10 days after the beginning of the differentiation protocol 100.000 cells were transduced at MOI 10 with a LV carrying the miRNA126 and the orange fluorescent protein under the control of Spleen Focus Formation virus (SFFV) promoter or with the LV carrying the miRNA let7b and the GFP under the same promoter. Endothelial cells were also co-transduced at M0110 with both LVs.

ECs Immunofluorescence and Flow Cytometry Analysis

For immunofluorescence staining, ECs were fixed with PFA 4% and stained following standard protocol. Primary antibodies included anti-FVIII (Green Mountain; 1:100), anti-vWF (1:100), anti-CD31 (1:100), anti-VEC (1:100). Secondary antibodies included Alexa Fluor 488 Goat anti-MouseIgG, Alexa Fluor® 546 Goat anti-Mouse IgG, Alexa Fluor® 546 Goat anti-Rabbit IgG and Alexa Fluor® 546 Donkey anti-Goat IgG. Nuclei were stained with 4,6-diamidino-2-phenylindole (DAPI).

Obtained ECs were characterized by flow cytometric analysis. Antibodies used and incubation conditions are reported in Table 2. For each sample, 1×105 events were acquired by FACS Calibur (BD). Data were analyzed by Windows Multiple Document Interface for Flow Cytometry (winMDI, v. 2.9; Joseph Trotter, The Scripps Institute).

In Vitro Tubulogenesis Assay

Pure Matrigel was added to each well of a 24-well tissue culture plate and allowed to solidify at 37° C. for 1 hour. Then 0.3 ml of a cell suspension containing 10⁵ endothelial cells in EB medium was placed on top of the Matrigel. Plates were incubated at 37° C., 5% CO2, and observed at 16 and 20 hours for observation of cellular organization into capillary-like structures.

LV Transduction of Endothelial Cells

Endothelial cells were transduced with LVs containing GFP under the control of endothelial specific promoters: Tie-2, VEC and Flk1. As positive control LV containing GFP under ubiquitous promoter (PGK) was used. As negative control LVs containing GFP under hepato- and myeloid-specific promoters were used (TTR and CD11b respectively). All LVs were used at MOI 10.

Animals and Procedures

Hemophilic NOD.Cg-Prkdcscidll2rgtm1Wjl/SzJ (HA-γNull), were generated in our laboratory and described by Zanolini et al., 2015, of 6-8 weeks of age mice were used for cell transplantation studies. Animals received 200 mg/Kg MCT in saline i.p. 24 hours before intraportal cell transplantation. Mice were anesthetized with isoflurane. For cell transplantation, 2×10⁶ of endothelial cells were injected into portal vein as previously described by Follenzi et al. in 2008 in 0.3 ml serum-free Dulbecco's Modified Eagle Medium (DMEM). Controls received serum-free medium.

For beads transplantation, cells were mixed with Cytodex 3 microcarriers in a ratio of 10·10⁶ cells mL-1 rehydrated microcarriers and injected intraperitoneally using a 20-gauge needle. Recipient animals were not treated with FVIII either prior to or subsequent to cell transplantation.

Immunostaining

Mouse tissues were fixed in 4% PFA for 2 h at 4° C., equilibrated in sucrose, and embedded in cryostat embedding medium. Cryostat sections of 4 μm thickness were blocked in buffer containing 5% goat serum, 1% BSA, and 0.1% Triton X-100 in PBS and incubated with rabbit anti-GFP (1:300) and with rat anti-mouse F4/80 (1:500) or mouse anti-human CD31 antibody (1:100). Sections were then incubated with Alexa Fluor 488-conjugated goat anti-rabbit IgG, with Alexa Fluor 546—conjugated goat anti-rat IgG and with Alexa Fluor 546—conjugated goat anti-mouse IgG using DAPI-Antifade for nuclear staining.

FVIII Activity

Plasma samples of transplanted mice and supernatants of LV-VEC-hBDDFVIII corrected ECs were analyzed for FVIII activity by aPTT. Standard curves were generated by serial dilution of pooled human plasma in hemophilic mouse plasma for aPTT assay. Results were expressed in percentage of correction. Bleeding assay was performed on anesthetized mice by cutting the distal portion of the tail at a diameter of 2.5-3 mm; the tails were then placed in a conical tube containing 14 ml of saline at 37° C. and blood was collected for 3′. Tubes were centrifuged to collect erythrocytes, resuspended in red blood lysis buffer (155 mM NH4Cl, 10 mM KHCO3, and 0.1 mM EDTA), and the absorbance of the sample was measured at wavelength 575 nm. Result was analyzed by comparing the amount of blood loss obtained from treated HA mice with WT and untreated HA mice serving as controls.

Generation of iPSCs from MNCs of Healthy and Hemophilic Donors

Peripheral blood MNCs were isolated from healthy and hemophilic donors and freshly and cultured (5 days) isolated cells were characterized.

Freshly isolated cells were:
CD3 (76%), CD11b (42%), CD14 (18%) and CD19 (8%) positive.

Meanwhile, after 5 days of culture, cells were mainly:
CD3+(86%), CD11b (19%) and CD19+(10%), thus were mainly lymphocytes and, in little part, monocytes.

Hemophilic MNC were corrected for FVIII expression with an LV carrying the B domain deleted form of FVIII, under the control of phosphoglycerate kinase (PGK) promoter (LV.PGK.hBDD-FVIII).

Then, cells were reprogrammed with a policystronic excisable LV carrying Oct4, Sox2 and Klf4 (LV.SFFV.OSK).

We obtained iPSCs from 2 of 5 healthy donors and 2 of 20 hemophilic patients (MNC-iPSCs and MNC-HA-iPSCs, respectively).

We obtained a mean of 2 good quality clones for each successful reprogramming. These clones reached high passages in culture (more than 50) and were positive at pluripotency assays.

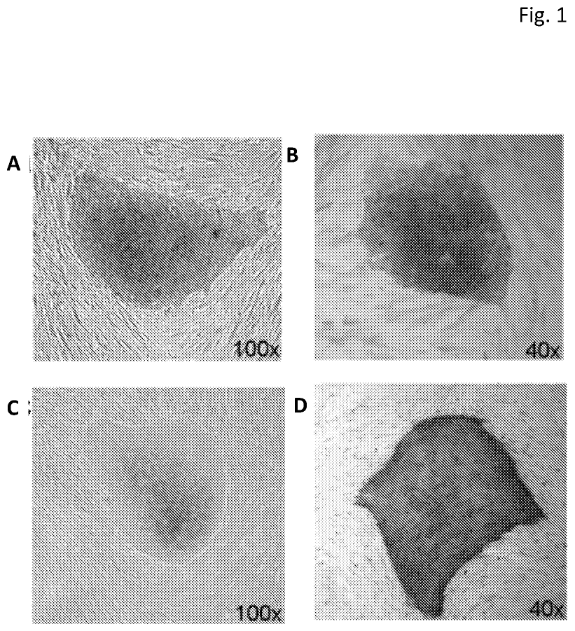

Healthy and hemophilic iPSCs appeared with ESC-like morphology, compact and with defined borders (FIGS. 1A and C). AP staining positivity (FIGS. 1B and D) displayed the reactivation of the enzyme. iPSCs expressed endogenous reprogramming factors, Oct4, Sox2 and Klf4, while RT-PCR, using primers specific for LV cassette, confirmed that exogenous factors were turned off.

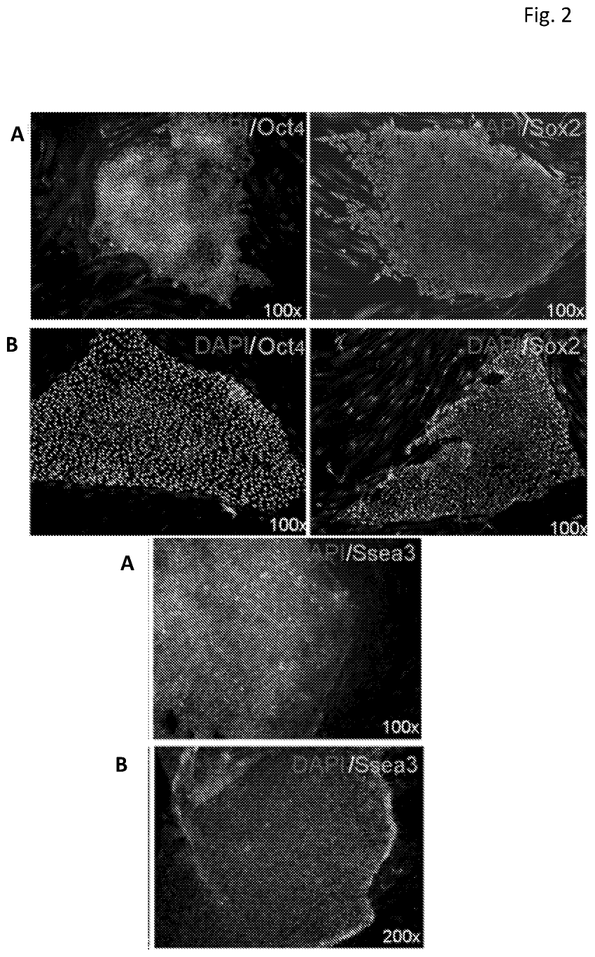

iPSCs expressed nuclear and surface pluripotent cells antigens, Oct4, Sox2 and SSEA-3 as shown by immunofluorescence analyses (FIGS. 2A and B). Moreover, an increase in telomeres length demonstrated the reactivation of telomerase complex.

However, the analysis of NANOG promoter methylation profile showed that only the 30% of analyzed CpG islands in NANOG promoter were unmethylated in iPSCs, suggesting that cells did not undergo at a complete reprogramming at the epigenetic level.

Interestingly, we obtained hemophilic iPSCs only from patient FVIII-corrected MNC. However, early-passage iPSCs did not express FVIII, although LV.PGK.hBDD-FVIII was integrated in transduced cells.

We supposed that PGK promoter was silenced during reprogramming process, indeed deep epigenetic changes occurred that probably influenced the promoter activity. To overcome this problem we decided to correct HA cells after the reprogramming and with a LV carrying the hBDD-FVIII under the VE-cadherin (VEC) promoter, specific of endothelial cells, our final cell target (LV.VEC.hBDD-FVIII).

Next, we evaluated the differentiation potential of the obtained iPSCs lines by in vitro embryoid body (EB) formation and differentiation assay.

RT-PCR analysis on EBs showed the expression of markers of the three germ layers (Nestin, NCAM and Otx2 for ectoderm; αSMA, Brachiury and Tbx6 for mesoderm; AFP, FOXA2 and SOX17 for endoderm). Moreover, EBs efficiently differentiated in adipogenic, osteogenic and chondrogenic cells.

Differentiation of Healthy iPSCs into Endothelial Cells.

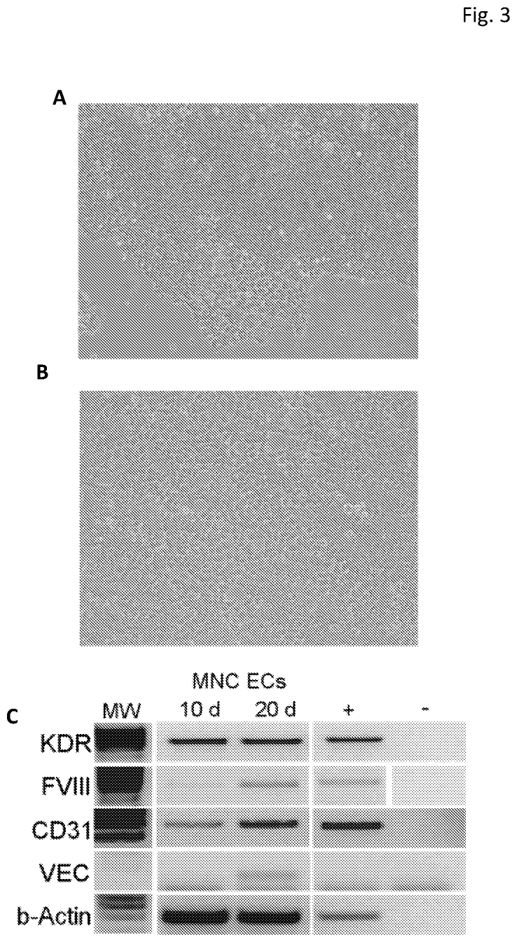

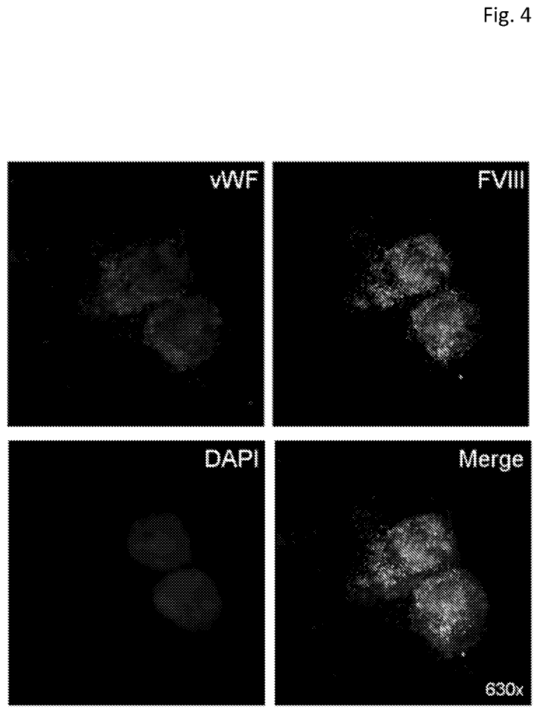

We differentiated MNC-iPSCs in endothelial cells through EBs formation induction using VEGF protocol, described in methods section. During the differentiation cells changed morphology acquiring the cuboidal shape typical of endothelial cells (FIGS. 3A and B). Analysis of gene expression showed an increase in endothelial markers such as KDR, CD31, VEC and, interestingly, FVIII (FIG. 3C). Immunofluorescence showed the costaining between FVIII and vWF (FIG. 4A). As further demonstration of endothelial differentiation, we transduced the obtained cells with LVs expressing GFP under the control of endothelial specific promoters Tie2 and Flk-1 and we used LV.PGK.GFP as positive control. The results showed that 60% of transduced cells expressed GFP under the control of Flk1 promoter, 50% under Tie2 promoter and 85% under PGK. These results confirmed that our cells started to differentiate in ECS but at the time of analysis the EC-differentiation was not complete and further experiments were necessary to address the best protocol to obtain endothelial cells. To investigate the engraftment capability of differentiated cells, we transplanted Flk-1-GFP+ cells by portal vein injection in MCT (200 mg/kg)—treated NOD-SCID HA mice. Immunofluorescence staining on liver sections evidenced the presence of GFP+ cells 96 hours after transplantation. By confocal analysis, we detected cells near blood vessels without a significant inflammatory response around transplanted cells as shown by F4/80 staining.

Genetic Correction of Hemophilic iPSCs and Differentiation into Endothelial Cells.

For differentiation of hemophilic iPSCs we used the VEGF protocol and we tested the efficiency of the BMP4 protocol, described in methods sections. Before EBs formation, MNC-HA-iPSCs were transduced with a LV carrying the hBDD-FVIII under the control of the endothelial-specific VEC promoter and, as control of VEC promoter activation during differentiation, with LV carrying GFP instead of FVIII. During differentiation cells changed morphology and started to express endothelial markers (KDR, Tie2, CD31) in a comparable way between the two protocols. Interestingly, LV.VEC.hBDD-FVIII transduced cells expressed FVIII. Analysis of telomeres length indicated a progressive shortening typical of differentiated cells. Nevertheless, VEC was not expressed, indicating that obtained endothelial cells did not reached a mature stage of differentiation and this was also confirmed by tubulogenesis assay. Indeed, MNC-iPSCs-derived ECs did not give rise to tubules network when plated in matrigel.

All together, these results on both healthy and hemophilic iPSCs differentiation suggested that we obtained a mixed population of differentiated cells, wherein not all cells reach the mature stage. Indeed, cells expressed early endothelial markers such as KDR, but low levels or no mature ones, such as VEC. Moreover, cells were not functional and were not able to form tubules. Given the fact that iPSCs from MNC were not reprogrammed at the epigenetic level, we suppose that the epigenetic memory of iPSCs made these cells resting to differentiation. Thus, we decided to generate iPSCs starting from CD34+ cells of peripheral blood, probably more prone to reprogramming.

Generation of iPSCs from Different Cell Sources of Healthy and Hemophilic Donors

The MNC reprogramming method reported above showed that we were able to generate iPSC. However, the efficiency of this method was not so high.

Therefore, we tried to improve the protocol by using a new reprogramming vector as first step.

Indeed, we reprogrammed cells using both LV.SFFV.OSK and a LV carrying, other than reprogramming factor, the miR 302/367 cluster (LV.SFFV.OSK.miR302/367), master regulators in the maintenance of hESC stemness.

Moreover, we considered different sources for the generation of iPSCs to evaluate the less invasive one for the donors from which obtain iPSCs with higher efficiency. Thus, we reprogrammed hemophilic patients skin fibroblasts. We generated iPSCs showing ES-like morphology, positive for alkaline phosphatase, expressing the endogenous factors, and in which the exogenous factors were silenced.

Immunofluorescence showed the expression of stem cells nuclear and surface antigens (Oct4, Sox2, Tra1-60, Ssea-3) and Nanog promoter analysis evidenced that 40% of CpG analyzed were in a unmethylated state.

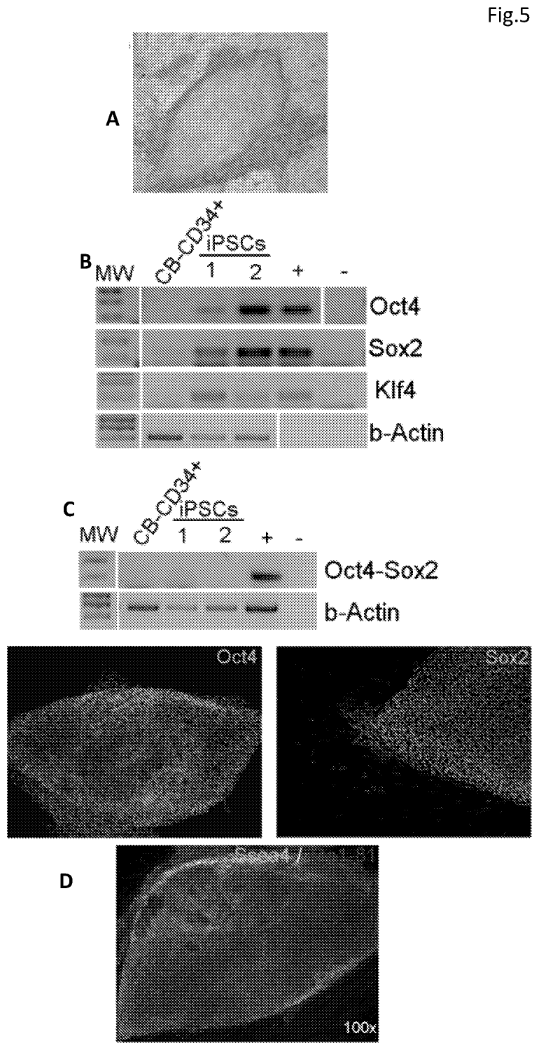

In parallel, we generated iPSCs from CD34+ cells isolated from cord blood and we obtained good quality clones as shown by AP staining (FIG. 5A), RT-PCR showing the expression of the endogenous factors (FIG. 5B), and the silencing of exogenous factors (FIG. 5C) and immunofluorescence (FIG. 5D).

Because CD34+ cells are an easy cell source to recover that can be obtained with less invasive techniques compared to fibroblasts, we decided to carry on the study using CD34+ cells from peripheral blood.

In 7 independent experiments (2 healthy donors, 1 heterozygous control and 4 hemophilic patients), we isolated a mean of 250.000 non-mobilized CD34+ cells from 20 ml of peripheral blood. After culture and expansion we obtained about 300.000-350.000 cells that we transduced with both reprogramming LVs. Colonies appeared about 20 days after reprogramming. We obtained about 20 clones from the reprogramming with LV.SFFV.OSK and more than 30 clones from LV.SFFV.OSK.miR302/367.

LV.SFFV.OSK-derived iPSCs degenerated rapidly.

Indeed, we were able to culture only four of these clones.

On the contrary, almost all from LV.SFFV.OSK.miR302/367-derived iPSCs reached advanced passages.

iPSCs colonies were picked basing on ESC-like morphology. We established a mean of 11 clones for each donors. In preliminary experiments, we used different MOI (5 and 10) to transduce cells to be reprogrammed and the yield of iPSC colonies did not rise as MOI was increased. Thus, we chose to use an MOI of 5 because the efficiency of transduction was enough so that the yield of iPSCs was adequate but low enough so that most iPSC clones had a copy number between 1 and 2.

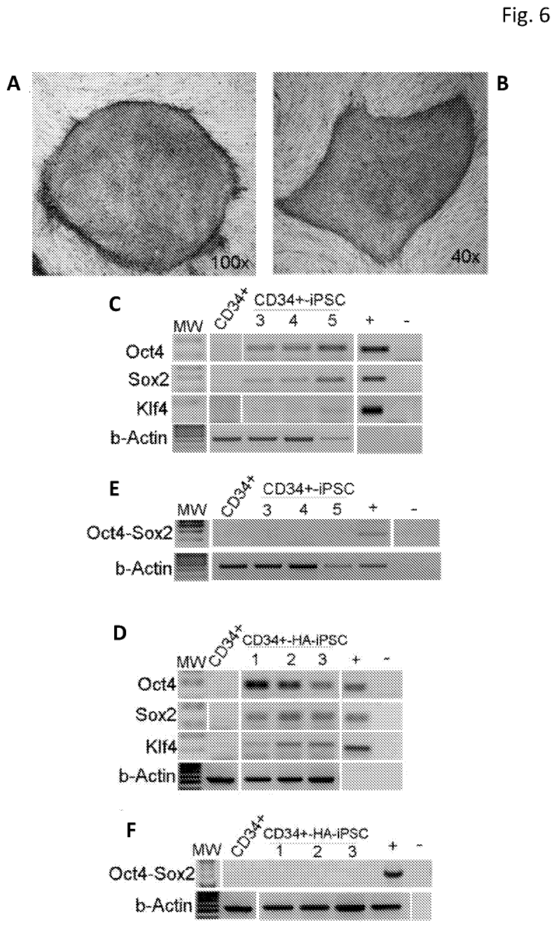

Indeed, when copy number was determined by Real Time analysis for the high quality iPSC clones, they showed a mean of 1.6 for healthy iPSCs and 1.4 for hemophilic iPSCs inserted copies/cell. We also analyzed iPSCs for pluripotency markers including AP positivity (FIGS. 6A and B), expression of endogenous and exogenous reprogramming factors (FIGS. 6C-D and E-F, respectively) and stem cells nuclear and surface antigens (Oct4, Sox2, Tra1-60, Ssea-3). iPSCs clones reached high passages (more than 50) maintaining a stable karyotype. Telomeres length increased between P5 and P20 (the longest passage analyzed) demonstrating the reactivation of telomerase complex. Moreover, NANOG promoter methylation profile showed that the 63% of analyzed CpG islands were unmethylated both in healthy and hemophilic iPSCs while in CD34+ cells from healthy and hemophilic donors the 92% and 95% respectively of sites were methylated.

These results showed that CD34+-derived iPSCs underwent to a complete reprogramming. Moreover, iPSCs were able to generate EBs which expressed three germ layers markers (Nestin, NCAM and Otx2 for ectoderm; αSMA, brachyury and Tbx6 for mesoderm; AFP, FOXA2 and SOX17 for endoderm) and were able to differentiated into adipogenic, osteogenic and chondrogenic cells.

Differentiation of CD34+-iPSCs into Endothelial Cells.

We differentiated CD34+-iPSCs into endothelial cell using both protocols described into “Material and methods” section.

During differentiation cell acquired an endothelial-like morphology. We extracted RNA at day 10 and 20 of differentiation. RT-PCR analysis showed an increase of endothelial markers, both early, such as KDR, and mature, like Tie-2, CD31 and VEC. Importantly for our goal, the mature differentiated cells showed an increased in FVIII expression. In particular, ECs differentiated by BMP4 protocol expressed endothelial markers in a comparable manner with HUVEC, used as positive control. We further confirmed a good endothelial gene expression was by FACS analysis. Indeed, 37% of ECs were positive at KDR staining, 40% for Tie-2, 65% for CD31 and 64% for VEC.

Interestingly, ECs expressed at higher levels mature markers CD31 and VEC than the earlier KDR, showing that the obtained cells were not progenitors but reached advanced stage of differentiation. To evaluate the activation of a pattern of endothelial specific promoters, we transduced ECs with LVs carrying GFP under the control of Flk-1, Tie-2 and VEC endothelial specific promoters (LV.Flk-1.GFP, LV.Tie-2.GFP, LV.VEC.GFP), the ubiquitous PGK promoter (LV.PGK.GFP) as positive transduction control. We used TTR hepatocytes specific and CD11bmyeloid specific promoters as negative control. FACS analysis showed that 72% of transduced cells were positive for GFP under Flk-1 promoter, 61% under Tie-2, 55% under VEC, while only 6% and 4% were positive under TTR and CD11 b promoters respectively. This result demonstrated the reactivation of endothelial promoters in terminally differentiated iPSC-derived ECs in a specific manner.

To confirm that we obtained mature differentiated cells we analyzed telomeres length and NANOG methylation profile comparing the ECs to the parental iPSCs. ECs showed a decrease telomeres length meaning the telomerase complex switching off, typical of differentiated cells. Methylation analysis indicated a 97% of methylated CpG islands at NANOG core promoter.

These data document that ECs can be efficiently derived from CD34+-iPSCs through EB induction differentiation methods.

Moreover, to assess that obtained ECs were able to form vessels-like structures, in vitro tubulogenesis assay was performed. iPSC-derived ECs, obtained from VEGF and BMP4 protocols, were able to form good tubules network after 16-18 hours in matrigel, demonstrating that they acquired the endothelial functionality.

Hemophilic CD34+-iPSCs were genetically corrected for FVIII expression and differentiated into endothelial cells.

Since BMP4 differentiation protocol seemed to give rise to differentiated cells that expressed endothelial markers better than VEGF protocol differentiated cells, HA-CD34-iPSCs were differentiated in ECs using the BMP4 one.

Before EBs formation induction, we genetically corrected iPSCs by transduction with LV.VEC.FVIII and LV.VEC.GFP was used as control. Then, EBs were formed from both transduced HA-CD34+-iPSCs and not transduced (NT) iPSCs. The protocol of differentiation was improved by transducing the cells with two different miRNAs: miRNA126 (SEQ ID NO: 19) and miRNA let7b (SE ID NO: 20). The miRNA126 is a well known endothelial specific miRNA involved also in angiogenesis, while the miRNA let7b is specifically expressed in microvascular endothelial cells.

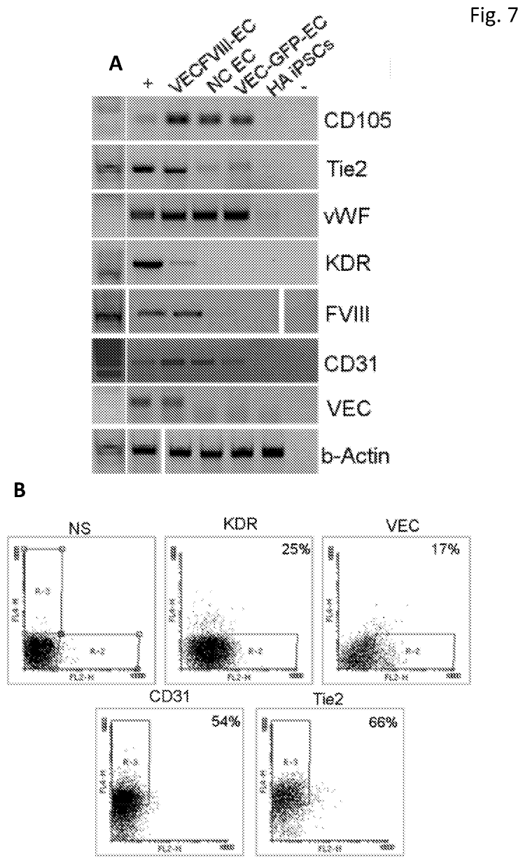

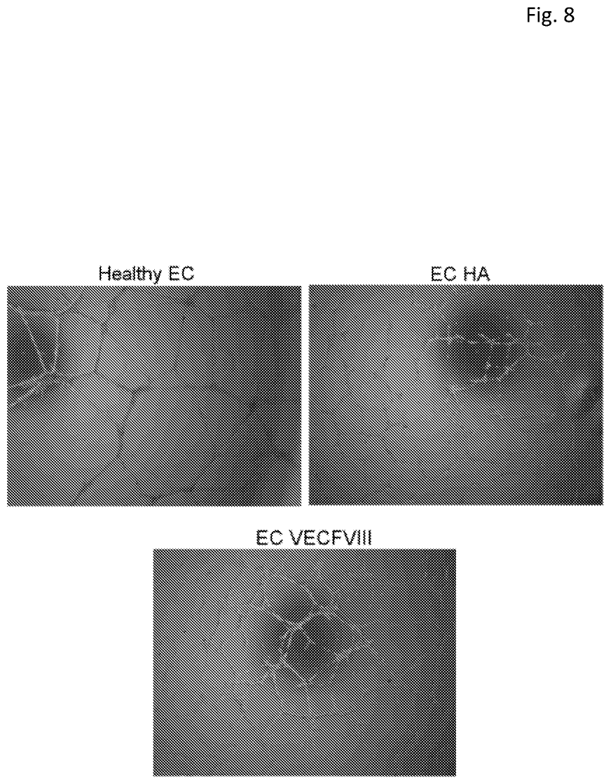

At day 10 of differentiation At day 10 of differentiation cells were transduced at MOI 10 with a LV carrying the miRNA126 and the orange fluorescent protein under the control of SFFV promoter or with the LV carrying the miRNA let7b and the GFP under the same promoter. Endothelial cells were also co-transduced at MOI 10 with both LVs. At day 20 of differentiation, 31% of ECs derived from VEC-GFP-iPSCs expressed GFP at FACS analysis, demonstrating that the endothelial specific promoter VEC was turned on during differentiation. Obtained VEC.FVIII, VEC.GFP and NT-ECs acquired endothelial-like morphology. RT-PCR (FIG. 7A) and FACS analysis (FIG. 7B) showed the expression of endothelial markers such as CD105, KDR, Tie-2, CD31 and VEC, both in transduced and not transduced ECs. Moreover, immunofluorescence staining revealed the expression of endothelial markers as CD31 and VEC staining highlighted the molecules distribution at cellular junction level. Moreover, VEC-FVIII-ECs expressed FVIII and, interestingly, vWF, another endothelial marker and FVIII carrier in the plasma. FVIII expression was confirmed by immunofluorescence staining that revealed FVIII presence near nuclei and, in smaller amount, in the cytoplasm of VEC.FVIII.ECs. As previously described for healthy ECs, telomeres length and NANOG methylation profile were analyzed and showed the mature stage of differentiation reached by HA CD34-iPSCs-derived ECs. Moreover, when ECs are co-transduced with the LVs carrying the miRNA 126 and let7b we observed an increase of expression, by RT-PCR, in VEC and Tie-2, suggesting that the protocol we developed and the use of the two miRNAs could induce a more mature stage of differentiation. Finally, we performed in vitro tubulogenesis assay and obtained interesting results. Indeed, NC-ECs started to form tubules but were not able to generate a complete network (FIG. 8). On the contrary, VEC-FVIII-ECs gave rise to a complex network and a higher percentage of cells seemed to take part at tubules formation (FIG. 8). Indeed, network generated by VEC-FVIII-ECs had a much more complex structure than formed by NC-ECs. Quantitative measurement of number of nodes, junctions, branches and segments and the length of branches in each well revealed a statistically significantly higher capillary formation in the net generated from corrected cells in comparison with that of non corrected. Moreover, when VEC-FVIII-ECs were co-transduced with the LVs carrying the miRNA 126 and let7b the complexity of the tubule network increased suggesting that they could be involved in the acquisition of the endothelial functionality. Moreover, migration assay showed that VEC-FVIII-ECs had a major motogenic potential respect non-corrected cells. Quantification of the total number of migrating cells revealed significantly (P<0.05) more migration of VEC-FVIII-ECs than NC-ECs (FIG. 8C). The number of migrating VEC-FVIII-ECs was 1.5 times that of NC-ECs. An increased trend healthy ECs migration was also visible respect the hemophilic ECs, but the increase was not statistically significant (FIG. 8C).

Finally, to assess if FVIII was not only produced but also efficiently secreted, aPTT analysis was performed on supernatant culture medium of VEC.FVIII, VEC.GFP and NC-ECs. This analysis revealed a shortening in aPTT of VEC-FVIII-ECs compared with NT- and VEC-GFP-ECs. All together, these results demonstrated that HA-CD34-iPSCs were efficiently genetically corrected by LV transduction. Then, during differentiation VEC promoter was switched on and FVIII was efficiently produced and secreted by HA-CD34-iPSCs-derived ECs at the end of differentiation. This result suggested that VEC-FVIII-ECs were genetically corrected and had the functionality of mature endothelial cells.

In Vivo FVIII Expression and Hemophilic Phenotype Correction after Transplantation of Genetically Corrected iPSCs-Derived ECs in a Mouse Model of HA

Since iPSCs were successfully differentiated into FVIII-expressing ECs we evaluated FVIII expression and secretion into our mouse model of HA. First, we assessed that ECs injected in peritoneal cavity survived and were able to secrete FVIII at therapeutic level. 10′7 GFP+ healthy iPSCs-derived ECs, hemophilic NC-ECs and VEC-FVIII-ECs were injected in association with microcarrier beads (n=4 each condition). Then FVIII activity was evaluated by aPTT assay 3 and 7 days after injection. At 3 days FVIII activity in mice injected with healthy iPSCs-derived ECs was 2.7±0.5% and with VEC-FVIII-ECs 4.9±1.3% while negative controls (beads only and HA NC-ECs) did not show FVIII activity. At 1 week after injection FVIII activity was maintained, indeed healthy iPSCs-derived ECs injected mice showed 2.1±0.4% FVIII activity and VEC-FVIII-ECs injected mice 5.0±0.8%. These results demonstrated that VEC-FVIII-ECs were able to secrete FVIII in vivo at therapeutic levels superior than healthy iPSCs-derived ECs.

Then immunofluorescence on recovered beads demonstrated that GFP+ cells were still present near the beads. These findings suggested that iPSCs-derived ECs survived and functionally secreted FVIII when injected in peritoneal cavity.

Following successful peritoneum injection, we transplanted 2×10\hat{ }6 healthy GFP+ iPSCs-derived ECs into the livers of monocrotalin (MCT)-conditioned γNull mice to evaluate the engraftment and proliferation of transplanted cells into liver of host mice. We observed engraftment and proliferation at 1 week, 4, 8 and 12 weeks after transplantation and we evaluated the presence of GFP+ cells by immunofluorescence on liver section. 1 week after transplantation GFP+ iPSCs-derived ECs engrafted in liver parenchyma without a significant immune response. 4 weeks after transplantation cells proliferated and repopulate about the 30% of transplanted mice liver. The costaining with human CD31 and the spindle shape morphology confirmed the endothelial phenotype of transplanted cells. The proliferation went on up to 3 months after transplantation, the longest time point analyzed. Transplanted cells maintained endothelial phenotype, expressing human CD31. Moreover, transplanted cells formed vessels-like structure in the host liver. These results demonstrated that healthy iPSCs-derived ECs were able to engraft and proliferate in mouse liver after transplantation. Thus, we transplanted VEC-FVIII-ECs in MCT-condition γNull-HA mice to evaluate FVIII secretion and phenotype correction. The engraftment was evaluated by FACS analysis of GFP+ and CD31+ cells percentage among liver non-parenchymal cells (NPC). The results showed that cells engrafted constituting about the 30% of NPC that were positive to the staining. On the contrary, no GFP+ hepatocytes were detected. The engraftment was also confirmed by immunofluorescence staining, indeed after 1 week GFP+ iPSCs-derived ECs engrafted in liver parenchyma without a significant immune response. 9 weeks after transplantation cells proliferated and repopulate about the 40% of transplanted mice liver. The costaining with human CD31 and human VE-cadherin confirmed the endothelial phenotype of transplanted cells. aPTT was performed 3, 6, 9 and 12 weeks after transplantation. The relative FVIII activity in mice transplanted with VEC-FVIII-ECs was 2.8±0.5% after 3 weeks and increased at 4.2±0.7% after 6 weeks remained stable at 9 and 12 weeks (4.6±0.3% and 4.7±0.7 respectively), while in mice transplanted with non-corrected cells no coagulation activity was detected. At 24 weeks, bleeding assay was performed and confirmed aPTT results. Indeed, mice transplanted with HA-ECs showed an increase in bleeding volume compared to VEC-FVIII-ECs transplanted mice. Taken together, these results demonstrated that hemophilic phenotype could be rescued by transplantation of ECs derived from HA-iPSCs and corrected by LV carrying FVIII under the control of endothelial-specific promoter VEC overtime up to 6 months confirming the capacity to form mature endothelial cells that after genetic correction where able to correct the bleeding phenotype of diseased animals.

1. Method for inducing pluripotent stem cells or embryonic stem-like cells comprising the following steps:
(i) having differentiated and/or somatic cells said cells selected from: fibroblasts, lymphocytes, mononuclear cells, and CD34+ cells said cells being isolated from an individual;
(ii) reprogramming said cells by transducing the cells with a viral vector comprising a DNA sequence codifying at least one transcription factor selected from Oct4, Sox-2, Klf4 and c-Myc, and/or at least one small RNA molecule, preferably selected from: miRNA 302 and/or 367;
(iii) culturing said reprogrammed cells in a medium specific for stem cells to isolate stable reprogrammed cell clones characterized by not more than 4 copies of the viral vector.

2. The method according to claim 1, wherein the mononuclear cells express at least one of the following markers: CD3, CD11b, CD14, and CD19; while the CD34+ cells are isolated from blood.

3. The method according to claim 1, wherein said individual is a healthy individual or a diseased individual, affected by a genetic disease comprising type A hemophilia.

4. The method according to claim 3, wherein the cells isolated from said diseased individual are genetically corrected by gene transfer or gene therapy.

5. The method according to claim 3, wherein the cells isolated from the type A hemophilia A affected individual are corrected, by gene therapy, by transducing into the diseased cells SEQ ID NO: 4, or any further gene involved in the coagulation cascade, wherein said transducing step comprises utilizing a viral vector comprising FVIII or its variant.

6. The method according to claim 5, wherein FVIII or its variants, or said any further gene involved in the coagulation cascade is under the expression control of VEC promoter or its variants SEQ ID NO: 8, or FVIII promoter or its variants, SEQ ID NO: 9-18.

7. The method according to claim 1, wherein the cells are activated before step (ii) by culturing them at least 48-70 hours till 4-10 days in a serum-free and/or xeno-free medium comprising cytokines selected from: IL-3, IL-6, IL-7, stem cell factor (SCF), GM-CSF, thrombopoietin (TPO) and FLT3-ligand (FLT3L).

8. The method according to claim 7, wherein the cytokines are used as a mixture.

9. The method according to claim 7, wherein the concentration of said cytokines ranges from 20 ng/ml to 100 ng/ml.

10. The method according to claim 8, wherein the concentration of the mixture of cytokines ranges between 5 and 25 ng/ml.

11. The method according to claim 1, wherein the viral vector is a lentiviral or retroviral vector.

12. The method according to claim 1, wherein the transducing step comprises is performed:
(i) inoculating the viral vector at least once at a multiplicity of infection (MOI) ranging from 5 to 100; and/or
(ii) on a cell amount ranging from 50.000 to 500.000; and/or
(iii) the viral vector has a titer ranging from 108 TU/ml to 1010 TU/ml; and/or
(iv) in a volume ranging from 50 μl to 500 μl.

13. The method according to claim 1, wherein before step (iii) the cells are cultured for at least 48-72 hours in a serum free medium specific for stem cells comprising a pre-mixed cocktail of recombinant human cytokines comprising: IL3, IL7, IL6, GM-CSF and combination thereof; and/or SCF, FLT3-ligand, TPO, IL3.

14. The method according to claim 1, wherein the step (iii) is performed on a feeder layer or in feeder free condition.

15. The method according to claim 1, wherein the step (iii) lasts for fibroblasts at least 6 weeks, for CD34+ at least 6 weeks.

16. Induced pluripotent stem cells or embryonic-like cells obtained/obtainable according to the method according to claim 1 characterized by:
Embryonic stem cell-like morphology compact with defined borders; and/or
Positive at alkaline phosphatase staining; and/or
Expressed stem cell nuclear and surface antigens, selected from the group consisting of: Oct4, Sox2, Klf4, Tra1-8 land Ssea-3/4; and/or
Unmethylated state of NANOG promoter; and/or
increase in telomeres therefore reactivation of telomerase complex; and/or
A normal karyotype; and/or
ability to differentiate all the cell types derived from the three germ layers.

17. Method for differentiating induced pluripotent stem cells or any embryonic stem like cells into endothelial cells, wherein said method comprises the following steps
(i) inducing the formation of embryo bodies starting from the induced pluripotent stem cells or embryonic-like cells by:
(ia) plating the cells in a medium specific for embryo bodies at a concentration ranging from 5 to 50, colonies/plate to obtain embryo bodies formation;
(ib) after about 48 h from step (ia), culturing the obtained embryo bodies in suspension in the medium specific for embryo bodies further comprising BMP4 at a concentration ranging from 5 to 40 mg/ml;
(ic) after about 90-100 hours from step (ia) further adding to the medium FGF specific for embryo bodies at a concentration ranging from 5 to 40 ng/ml;
(id) after about 130-150 hours from step (ia) seeding the obtained embryo bodies on a gelatin coated plate in a medium specific for embryo bodies comprising: FGF at a concentration ranging from 5 to 40 ng/ml; and/or VEGF at a concentration ranging from 30 to 70 ng/ml;
(ie) after about 180-200 hours from step (ia) replacing the medium specific for embryo bodies with a medium including VEGF at a concentration 30-70 ng/ml until the end of culturing 20 days;
(ii) collecting the culture cells wherein said cultured cells are endothelial cells.

18. Endothelial cells obtained by the method of claim 17.

19. A pharmaceutical composition comprising the induced pluripotent stem cells of claim 16 and/or the (differentiated) endothelial cells of claim 18 and at least one further pharmaceutical acceptable agents, such as carriers, diluents, adjuvants, growth factors.

20. A method for treating a genetic disease with the induced pluripotent stem cells of claim 16, or the (differentiated) endothelial cells of claim 18, or the pharmaceutical composition of claim 19, wherein said genetic disease comprises type A hemophilia.
